(19) United States
(12) Patent Application Publication
(10) Pub. No.: US 2024/0009272 A1
(43) Pub. Date: Jan. 11, 2024

(54) METHODS, SYSTEMS, AND COMPOSITIONS FOR LEGUME-BASED PRODUCTION OF THERAPEUTIC PROTEINS AND THERAPEUTIC MEDICAL MATERIALS

(71) Applicant: Arizona Board of Regents on Behalf of The University of Arizona, Tucson, AZ (US)

(72) Inventors: Eliot M. Herman, Tucson, AZ (US); Monica Schmidt, Tucson, AZ (US); Marvin J. Slepian, Tucson, AZ (US)

(21) Appl. No.: 18/174,158

(22) Filed: Feb. 24, 2023

Related U.S. Application Data
(63) Continuation-in-part of application No. 16/623,187, filed on Dec. 16, 2019, now abandoned.
(60) Provisional application No. 62/521,161, filed on Jun. 16, 2017.

(51) Int. Cl. A61K 38/18; A61K 36/48; A61K 38/28; A61K 38/39; A61K 38/17; A61K 45/06; A61K 9/14

(52) U.S. Cl. CPC A61K 38/1808; A61K 36/48; A61K 38/28; A61K 38/39; A61K 38/1709; A61K 45/06; A61K 9/14

(57) ABSTRACT

Compositions and materials comprising transgenic soybeans expressing a codon-optimized gene encoding of the hEGF protein. Using these methods, the production of hEGF is sufficient and cost-effective, thus providing a more economical way to produce medical materials comprising said protein.

### CROSS REFERENCE

This application is a continuation-in-part and claims benefit of U.S. patent application Ser. No. 16/623,187 filed Dec. 16, 2019, which is a 371 and claims benefit of PCT Application No. PCT/US2018/038096 filed Jun. 18, 2018, which claims benefit of U.S. Provisional Application No. 62/521,161 filed Jun. 16, 2017, the specifications of which are incorporated herein in their entirety by reference.

### GOVERNMENT SUPPORT

This invention was made with government support under Grant No. R21 DK094065 awarded by National Institutes of Health. The government has certain rights in the invention.

### REFERENCE TO AN ELECTRONIC SEQUENCE LISTING

The contents of the electronic sequence listing (UNIA 17.30 PCT US CIP Sequence Listing.xml; Size: 13,059,279; and Date of Creation: Feb. 23, 2023) is herein incorporated by reference in its entirety.

### FIELD OF THE INVENTION

The present invention relates to protein production in legumes, more particularly to the production of various proteins, such as therapeutic proteins in legumes, and further to the production of legume-based materials and therapeutic constructs comprising therapeutic proteins.

### BACKGROUND OF THE INVENTION

Protein and peptide therapeutics have emerged as an increasingly vital therapy for a wide range of conditions. For example, in diabetes, diabetic foot ulceration results in acute and chronic wounds that are difficult to heal without growth factor administration, often resulting in amputation. In children, necrotizing enterocolitis often leads to malabsorption and requires growth factor administration for repairing damaged intestinal epithelia. While growth factors have increasingly been brought forward as biological therapeutic agents, their production, processing, and delivery to the recipient (e.g., human, animal, etc.), remains complex, expensive, and cumbersome.

The present invention features methods, systems, and compositions for producing proteins (e.g., therapeutic proteins) in legumes. Legumes may include but are not limited to alfalfa, clover, mesquite, tamarind, carob, peas, beans, peanuts, or other legume nuts, lentils, and soybeans. The proteins produced by the legumes may be used for delivery in animals such as humans or other species. As a non-limiting example, the present invention features transgenic legumes expressing a gene to produce a growth factor, e.g., EGF, FGF, PDGF, VEGF, IGF, HSP, TGF-alpha, TGF-beta, etc., or bioregulatory or therapeutic proteins such as insulin, fibronectin or HIF-1 alpha.

Methods of production of said proteins (e.g., therapeutic proteins) may feature gene splicing into legumes. While the present invention discloses EGF production in soybeans, the specific genetic manipulative methodology described in the present invention is broadly applicable to a range of proteins (e.g., therapeutic proteins, e.g., growth factors) and a range of legumes.

The present invention also features methods to remove at least a portion of mutagenic and/or inflammatory elements of the soybean plant, e.g., selectively clone out or reduce expression of specific host (plant) proteins or factors that may be inflammatory when applied as a concomitant unprocessed therapeutic to an individual. For example, some proteins expressed in soy may ultimately be inflammatory and/or allergenic to a human. Without wishing to limit the present invention to any theory or mechanism, it is believed that removing mutagenic and/or inflammatory elements of the soybean plant, e.g., when applied to humans or animals, will not reduce the efficacy of the therapeutic protein.

The present invention also features methods for delivering a protein (e.g., growth factor) from the plant (legume) source. Conventional methodologies may take a spliced in, transduced, and translated human protein therapeutic raised in a cross-kingdom source (e.g., a plant) and go through a range of processing steps to extract the human therapeutic. Without wishing to limit the present invention to any theory or mechanism, a potential downside or limitation of this type of approach may include a reduction in yield, a risk of protein denaturation, storage, and stability issues, and/or an ultimate reduction in efficacy and possible safety. The present invention features a range of methods and products for the delivery of the protein (e.g., therapeutic protein) using the plant substance as a delivery vehicle. The entire raw plant may be processed or any other part or combination of parts. Methods of fabrication of raw materials include but are not limited to grinding, particulating, pulverizing, morcellating and other alteration of mass means. In addition, the non-protein yielding elements of the plant may similarly be processed by the above techniques, stripped, sub-fractionated, or otherwise extracted to yield materials with a range of material properties and stiffness.

The present invention also features a range of therapeutic protein products that may be fabricated from these base materials into novel configurations with novel properties. As an example, these raw base materials may then be fabricated via a range of processing/manufacturing techniques, including film formation—either alone or with intermixed adjuvants and binders, including natural and synthetic gelation materials, e.g., PEG, PEG-lactide, Plutronics, Tetronics, Carbopol, Eudragits, Gelatins (see, for example, U.S. Pat. No. 6,290,729, the disclosure of which is incorporated herein by reference in its entirety), spray drying, drop casting, spin casting, extrusion, electrospinning, low-temperature thermoforming, micro- and nano-particle or micro- and nano-capsule formation and/or other related formation techniques. These materials may then be processed and mixed with other constructive elements to form specific delivery products that may be utilized for topical, dermal, or enteral use. As an example of a novel construct, soybean bulk plant shaft material in combination with raw soybean containing EGF has been micro pulverized, formed into a slurry, and electrospun to yield a matte bandage and gauze, which may be applied directly to a wound such as a diabetic foot ulcer, a post-surgical incision site, or a non-healing sternal wound or mediastinitis site. Similarly, constructed products may be utilized in the animal domain, e.g., in significant wounds to a racehorse, wherein non-healing wounds may result in animal euthanasia. Another example is the fabrication of an endoluminal stent-like construct that may be applied by balloon catheter endoluminally to the G.I. tract for local ulceration, either in the stomach or anywhere from the mouth to the anus. Novelty in the construct includes adding in non-active plant elements or synthetic elements that may be hygroscopic, leading to reduction of edema, removing of fluid from weeping wounds, and otherwise drying up a supportive wound bed. In the non-therapeutic elements, indicators markers and sensors may also be admixed to provide active probing and feedback information as to wound status and progression. In another formulation, non-allergenic soy may be admixed with binders as above and locally injected in the more superficial layers of skin to yield depots for the local release of the human therapeutic agent. Also admixed in these constructs, both topical and intradermal or enteral, may be a range of synergistic medications that may be anti-inflammatory, anti-infective, or anesthetic for pain reduction.

The present invention shows the feasibility of using plants as a biofactory to produce therapeutic agents for a delivery platform.

Without wishing to limit the present invention to any theory or mechanism, it is believed that there are a number of ways to eliminate inflammatory proteins, e.g., using genetic engineering approaches, selective breeding using mutants from collections, conventional gene silencing via suppression, CRISPR mediated mutation, natural, spontaneous mutation (e.g., used for a triple null soybean), etc. Using such a platform, the expression of a variety of different proteins including but not limited to EGF could be engineered.

See references Hemendrasinh J Rathod and Dhruti P Mehta. “A Review on Pharmaceutical Gel.” International Journal of Pharma and Drug Development (2016): 25-36; and Ganapathy et al., J Pharm Bioallied Sci. 2012 August; 4(Suppl 2): S334-S337.

### SUMMARY OF THE INVENTION

The present invention features methods, systems, and compositions for plant-based production of proteins (e.g., therapeutic proteins), peptides (e.g., therapeutic peptides), and therapeutic materials. For example, the present invention features transgenic legumes expressing a protein, the protein being an animal protein (e.g., a human protein, a human growth factor, etc.). In some embodiments, the protein is a therapeutic protein. In some embodiments, the transgenic legume is a soybean, a lentil, a bean, a pea, or a peanut. In some embodiments, the protein is a growth factor. In some embodiments, the protein is an antibody. In some embodiments, the animal protein is a human protein. In some embodiments, the transgenic legume is a non-allergenic legume. In some embodiments, the transgenic legume is a non-allergenic soybean.

The present invention also features medical materials comprising an animal protein derived from a transgenic legume, according to the present invention, and at least a portion of the transgenic legume that produced said protein. In some embodiments, the material is for epidermal or dermal application. In some embodiments, the material comprises gauze or a bandage. In some embodiments, the material is constructed by spin-coating, drop casting, spin casting, extrusion, electrospinning, film formation, spraying, spray drying, drop casting, spin casting, extrusion, electrospinning, low-temperature thermoforming, micro-particle formation, nano-particle formation, micro-capsule formation, nano-capsule formation, or a combination thereof. In some embodiments, the medical material reduces inflammation. In some embodiments, the material further comprises a non-active plant element. In some embodiments, the material further comprises a synthetic element. In some embodiments, the element comprises an excipient or adjuvant. In some embodiments, the excipient or adjuvant comprises a colloidal binder, gelatin, polyethylene glycol (PEG), PEG-lactide, Plutronics, Tetronics, Carbopol, Eudragits, or a combination thereof. In some embodiments, the element is hygroscopic. In some embodiments, the element is hydrophobic. In some embodiments, the construct contains a hydrogel, aerogel, or organogel element or material or a combination thereof or other gel or gellant materials similar to those discussed in Rathod and Mehta 2016. In some embodiments, the material further comprises a marker or sensor, or means of detection. In some embodiments, the sensor is for providing feedback information as to the status of the topical condition. In some embodiments, the sensor or marker is for pH detection or indication. In some embodiments, the sensor or marker is for detecting infection. In some embodiments, the material further comprises a medication. In some embodiments, the medication is an anti-inflammatory medication, an anti-bacterial medication, an antimicrobial medication, an antifungal medication, an anti-infective medication, an anesthetic medication, or a combination thereof. In some embodiments, the material further comprises a perfumant. In some embodiments, the material further comprises a compound or compounds for reducing odor. In some embodiments, the material further comprises non-allergenic soy. In some embodiments, the material may contain and/or deliver a cell or cell product. As an example, the material may deliver live, dead, or attenuated epithelial cells, platelets, or white blood cells; in some embodiments, the material may contain or deliver a cell product or constituents such as platelet-rich plasma or extract; in some embodiments, the material may contain or deliver a viral vector, gene, plasmid, episome or bacteriophage, siRNA, aptamer, and the like genetic material.

The present invention also features a method of treating a topical condition, wherein the method may comprise applying to the topical condition a medical material according to the present invention.

One of the unique and inventive technical features of the present invention is the use of proteins (e.g., therapeutic proteins) produced from transgenic legumes (e.g., transgenic soybeans). Without wishing to limit the invention to any theory or mechanism, it is believed that the technical feature of the present invention advantageously provides cost-effective ways to produce said proteins (e.g., therapeutic proteins) for use in medical applications. When compared to biotech methods with expensive reactors and complicated processes, the cost here is really simple farming. None of the presently known prior references or work has the unique, inventive technical feature of the present invention.

The present invention also features compositions comprising the animal protein according to the present invention. In some embodiments, the composition comprises soymilk.

The present invention also features methods of harvesting a recombinant protein expressed in a transgenic legume. In some embodiments, the method comprises processing an entire plant of the transgenic legume. In some embodiments, processing the entire plant comprises grinding. In some embodiments, processing the entire plant comprises micro pulverizing.

The present invention also features methods and compositions for producing epidermal growth factor (EGF) (e.g., human EGF) in soybean seeds. For example, the present invention also features a method of producing human epidermal growth factor (hEGF). The method may comprise expressing a protein encoded by SEQ ID NO: 1 (a codon-optimized gene for EGF expression) in a transgenic soybean comprising a transgene according to SEQ ID NO: 1 (see FIG. 7 for SEQ ID NO: 1). In some embodiments, the method further comprises purifying said hEGF and/or reconstituting said hEGF in a solution. In some embodiments, the solution comprises soymilk.

As such, the present invention also features a nucleic acid according to SEQ ID NO: 1. The present invention also features a protein encoded by a nucleic acid according to SEQ ID NO: 1. The present invention also features a transgenic soybean expressing SEQ ID NO: 1. The present invention also features a soymilk composition comprising soybean-derived human epidermal growth factor (hEGF).

As previously discussed, the methods of the present invention are such that one does not necessarily have to process the soybean or other legume to extract the protein, which could reduce yield or risk damaging or denaturing the protein. Methods of the present invention may feature grinding and optionally processing the entire plant (e.g., not necessarily just the bean) to create a range of constructs, including spun gauze, bandages, and injectable intradermal fields of local depots. In addition, this may be used in other open lumens, including the sinus for sinusitis, the mouth for oral ulcers, and anywhere in the enteral tract. This may be formed into an enteral stent for local ulceration and/or local delivery of other protein therapeutics. The present invention also features antibody production. Antibodies may be used for a range of indications, including but not limited to inflammatory bowel disease.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

The features and advantages of the present invention will become apparent from a consideration of the following detailed description presented in connection with the accompanying drawings in which:

FIG. 1A shows a schematic diagram of seed-specific gene expression cassettes, e.g., to direct ShEGF. For example, a synthetically produced codon-optimized hEGF gene with an ER signal added to the amino terminus driven by glycinin regulatory elements was transformed via biolistics into somatic soybean embryos. GLY refers to the glycinin promoter. LEA refers to late embryonic abundant protein promoter. KDEL (SEQ ID NO: 45) is located on the C-terminus. The presence of ER signal peptide/retention tag may enhance the yield of EGF accumulated in the soybean seeds.

FIG. 1B shows ELISA quantification for both the detection and amount of hEGF in total soluble dry seed protein extract from 7 ShEGF transgenic soybean lines. Independent homozygous lines 1, 3, 4, 5, 6, 11, and 13 were detected to contain hEGF up to 129 μg EGF/g seed compared to undetectable amounts in non-transgenic control (Wt). Values shown are mean+/− standard error (n=3).

FIG. 2 shows an analysis of total soluble protein by one-dimensional gel electrophoresis of hEGF expressing transgenic soybean seeds. Proteins from 3 independent homozygous EGF transgenic soybean lines (3, 4, 5) were extracted and compared to seed extracts from non-transgenic (Wt) and commercially available hEGF standard (STD+). M marker, kDa kilobases.

FIG. 3 shows an immunoblot of enriched small molecular weight soluble protein extracted from dry transgenic ShEGF soybean seeds. Protein extracts from two independent homozygous lines (5 and 4) are compared to both non-transgenic (Wt) and commercially available EGF standards (STD+). EGF was detected using an EGF-specific antibody and an indirect secondary antibody coupled to alkaline phosphatase. M marker; kDa kilodalton.

FIGS. 4A and 4B show mass spectroscopy data to detect the presence of EGF peptides (SEQ ID NO: 35) in transgenic EGF soybean seeds. FIG. 4A shows the coverage of peptides detected in both commercially available EGF (top) and from transgenic soybean seeds (bottom) using both trypsin (solid) and non-trypsin peptides (hatched). FIG. 4B shows raw spectra data depicting the amino acid sequence CNCVVGYIGER (SEQ ID NO: 36) detected from a low molecular weight enriched soluble dry seed protein extract from EGF transgenic soybean.

FIGS. 5A, 5B, and 5C show soybean produced EGF displayed comparable bioactivity to commercially available EGF. FIG. 5A shows that soybean-produced hEGF induces rapid phosphorylation of Hela cell EGFR. Serum-free (SF) media and SF media with soymilk alone do not induce EGFR phosphorylation and degradation. Soy milk from seeds producing ShEGF added at different concentrations (0.1, 0.05, 0.025 μg/ml) induced concentration-dependent EGFR degradation comparable to the effect of rhEGF. Serum-free media and serum-free media with non-transgenic soybean soymilk (negative controls) showed no effect on inducing pEGFR. In contrast, soymilk from ShEGF soybeans given at different concentrations (0.1, 0.05, 0.025 μg/ml) induced pEGFR comparable to control rhEGF. pAKT indicates the functional activation of EGFR. Lamin 1 was used as a loading control. FIG. 5B shows exogenous commercial rhEGF and ShEGF induces internalization and degradation of EGFR in Hela cells, shown by a decrease in abundance assayed by immunoblot. The results shown demonstrate that soymilk alone has no intrinsic bioactivity with respect to EGFR abundance. The rhEGF is not degraded in soymilk over 24 hours, having the same bioactivity as control recombinant rhEGF. Ctrl-SF media alone. Soy EGF and rhEGF are at 0.1 μg/ml. Lamin 1 was used as a loading control. FIG. 5C shows an immunohistochemical assay of Hela cells showing that ShEGF induces internalization of the EGFR comparable to that from control rhEGF. In FIG. 5C, the cells were first treated with soy/EGF or human EGF for 6 hours, fixed, and then immunostained with EGFR antibody overnight. Both EGFR and DAPI were stained.

FIG. 6 shows differences (insignificant differences) between non-transgenic soybean seeds and the ShEGF transgenic seeds.

FIG. 7 shows the human EGF DNA sequence (SEQ ID NO: 23) and Amino Acid sequence (SEQ ID NO: 35), and the optimized EGF DNA sequence for soybean transformation (SEQ ID NO: 1) and Amino Acid sequence (SEQ ID NO: 12).

FIG. 8A shows an expression cassette targeted to the endoplasmic reticulum (ER), thus said expression cassette comprises a 5′ ER sequence (underlined; SEQ ID NO: 24), and the protein sequence to encode was an epidermal growth factor (hEGF) protein (bolded; SEQ ID NO: 35).

FIG. 8B shows an expression cassette comprising a 5′ ER sequence (underlined; SEQ ID NO: 24), an epidermal growth factor (hEGF) protein sequence (bolded; SEQ ID NO: 35), and a KDEL sequence (italicized; SEQ ID NO 25).

FIG. 8C shows the nucleotide sequence for the expression cassette (SEQ ID NO: 37). The NOTI restriction site was used for ease of cloning (shaded; SEQ ID NO: 38). The expression cassette comprises the endoplasmic reticulum (ER)-directed 5′ sequence (underlined; SED ID NO: 39), the codon-optimized soybean EGF protein (SEQ ID NO: 40, bolded), the ER-retention portion (i.e., the KDEL sequence; SEQ ID NO: 41; italicize). Note the ER-retention portion was not used on all constructs.

FIGS. 9A, 9B, 9C, 9D, 9E, and 9F show various constructs of the present invention; such as continuous or microporous construct (FIG. 9A); macroporous construct (FIG. 9B); vacuous, discontinuous or holey construct (FIG. 9C); fibrous or filamentous construct (FIG. 9D); construct with intra or subdermal penetration (FIG. 9E); constructs with intra or subdermal penetration (FIG. 9F).

FIGS. 10A, 10B, and 10C show applications of compositions of the present invention; such as therapeutically applied to wound topically (FIG. 10A); therapeutic applied to wound sub- or intra-dermally (FIG. 10B); a combination of topical and sub- and intra-dermal application (FIG. 10C).

### DETAILED DESCRIPTION OF THE INVENTION

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

Additionally, although embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. Moreover, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described herein.

As used herein, the singular forms “a,” “an,” and “the” are intended to include the plural forms as well unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

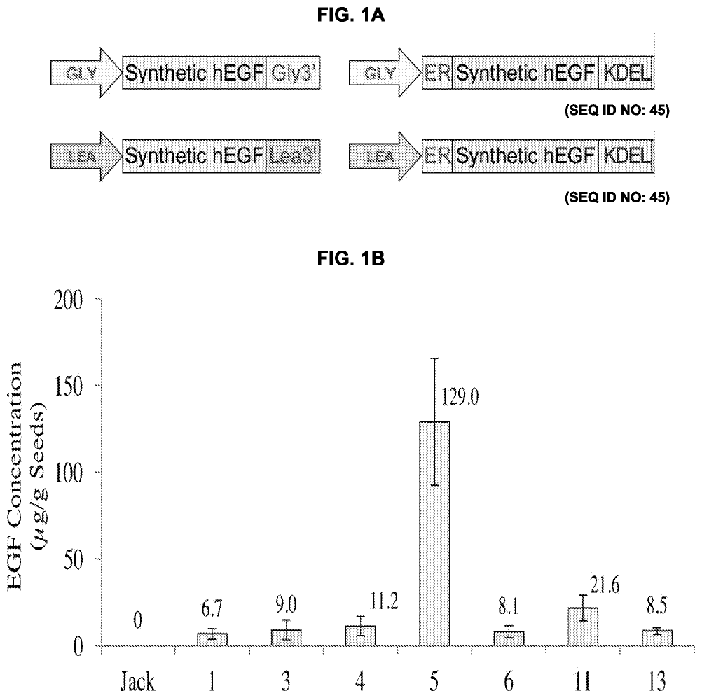

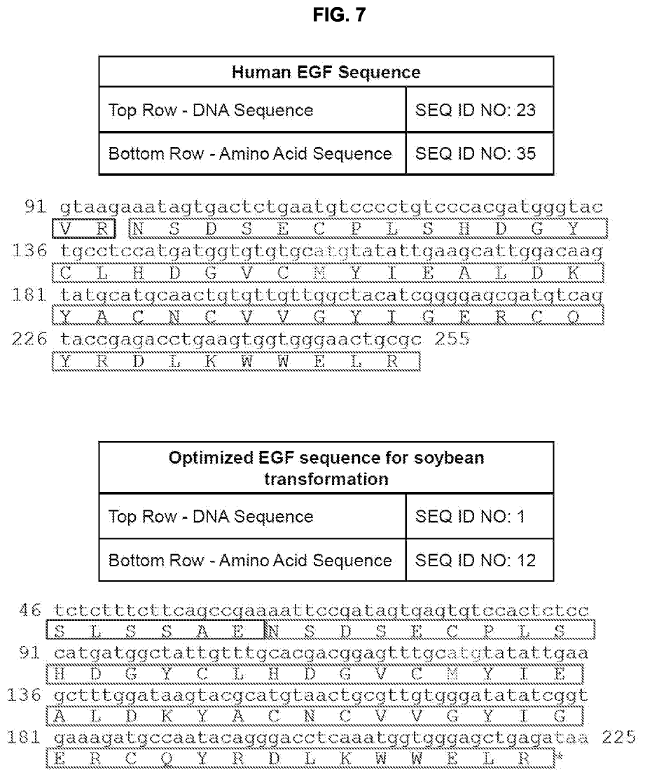

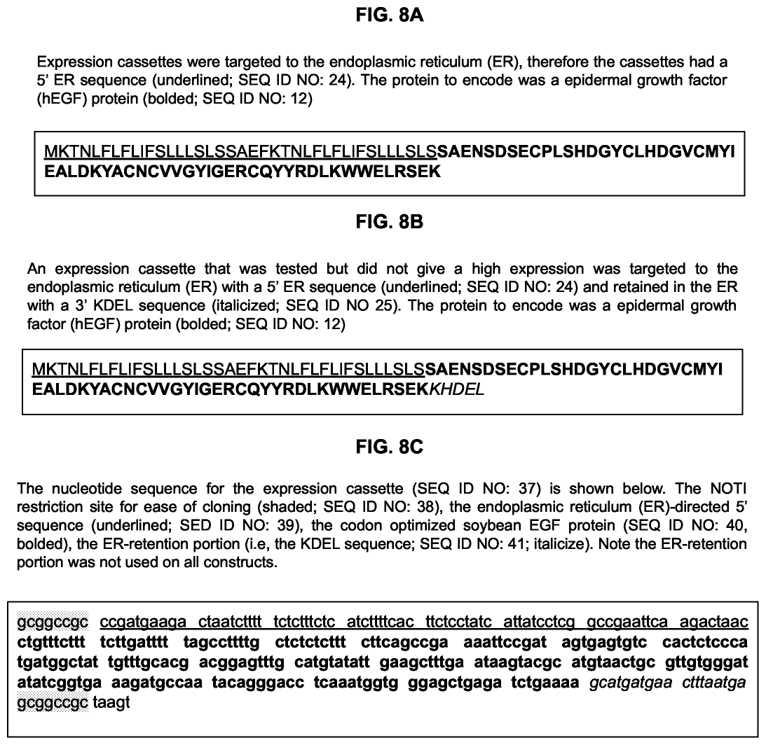

Referring now to FIG. 1A-10C, the present invention features compositions and methods for producing proteins (e.g., therapeutic proteins) and/or peptides (e.g., therapeutic peptides) in legumes (e.g., soybeans) and using said therapeutic proteins for the construction of medical materials.

As used herein, a “peptide” may refer to short chains of amino acids. In some embodiments, a peptide refers to a chain of amino acids that is 50 amino acids or less. As used herein, a “protein” may refer to long chains of amino acids and may be made up of multiple peptide subunits. In some embodiments, a protein refers to a chain of amino acids that is greater than 50 amino acids. In some embodiments, the peptides described herein may be made via direct transcription that has an element leading to termination, e.g., generating an incomplete protein. In other embodiments, the peptides described herein may be mixed in to create a physical blend, could be encapsulated, could be linked to other proteins in the medical material, or other constituents, either non-covalently or covalently.

The present invention features a medical material comprising an animal or human protein derived from a transgenic legume or at least a portion of the transgenic legume. In some embodiments, the transgenic legume produces said animal or human protein. In some embodiments, the present invention features a medical material comprising at least one animal or human protein derived from a transgenic legume or at least a portion of the transgenic legume. In some embodiments, the transgenic legume produces said animal or human protein. In some embodiments, the present invention may feature a medical material comprising a soluble, bioactive, human epidermal growth factor (hEGF) protein produced by said transgenic legume or at least a portion of the transgenic legume. Non-limiting examples of legumes may include but are not limited to alfalfa, clover, mesquite, tamarind, carob, peas, beans, peanuts, or other legume nuts, lentils, and soybeans.

In some embodiments, the present invention may also feature a medical material comprising an animal or human protein (e.g., at least one animal or human protein) derived from a transgenic soybean or at least a portion of the transgenic soybean. In some embodiments, the transgenic soybean produces said animal or human protein. In other embodiments, the present invention may feature a medical material comprising a human epidermal growth factor (hEGF) protein produced by a transgenic soybean plant or at least a portion of the transgenic soybean plant. In further embodiments, the present invention further features a medical material comprising a soluble, bioactive human epidermal growth factor (hEGF) protein produced by a transgenic soybean plant or at least a portion of the transgenic soybean plant.

In some embodiments, the aforementioned medical material may further comprise at least one soybean protein derived from a transgenic soybean or at least a portion of the transgenic soybean. The soybean protein may comprise 7S, 11S, or a combination thereof.

The present invention may further feature a medical material comprising at least one soybean protein derived from a transgenic soybean or at least a portion of the transgenic soybean. In some embodiments, the at least one soybean protein comprises 7S, 11S, or a combination thereof. The medical material may further comprise an animal or human protein derived from the transgenic soybean or at least a portion of the transgenic soybean, wherein the transgenic soybean produces said animal or human protein.

The present invention may also feature a medical material comprising at least one soybean protein and an animal or human protein derived from a transgenic soybean or at least a portion of the transgenic soybean, wherein the transgenic soybean produces said animal or human protein. In some embodiments, the present invention may also feature a medical material comprising at least one soybean protein and at least one animal or human protein derived from a transgenic soybean or at least a portion of the transgenic soybean, wherein the transgenic soybean produces said animal or human protein. In some embodiments, the at least one soybean protein comprises 7S, 11S, or a combination thereof.

As used herein, a “medical material” may refer to a soy-based material. In some embodiments, a medical material is a soy-based material that is mechanically modified (e.g., by grinding, pulverizing, or otherwise morselizing said soy-based material.

In some embodiments, the medical material described herein may be used for therapeutic applications. For example, the medical material may act as an active hygroscopic gauze material to soak up serous fluid, etc. In some embodiments, the medical material may comprise EGF for wound healing. In some embodiments, the medical material may have the EGF or a peptide added to said material, and similarly, a bandage is formed.

In some embodiments, the medical material forms a medical construct. In some embodiments, the medical constructs described herein may be used for therapeutic or diagnostic purposes.

As used herein, a “medical construct” may refer to materials that are consumable, expendable, disposable, or non-durable and that are used for the treatment and/or diagnosis of an illness, injury, or condition.

In some embodiments, the transgenic legume is a non-allergenic transgenic legume. In some embodiments, the transgenic soybean plant is a non-allergenic soybean plant.

In some embodiments, the protein produced by the transgenic legume (e.g., a transgenic soybean) is soluble. In some embodiments, the protein produced by the transgenic legume (e.g., a transgenic soybean) is bioactive. In some embodiments, the transgenic legume (e.g., a transgenic soybean) produces an epidermal growth factor (EGF) protein. In some embodiments, the transgenic legume (e.g., a transgenic soybean) produces a human epidermal growth factor (hEGF) protein. In some embodiments, the transgenic legume (e.g., a transgenic soybean) produces a soluble, bioactive, endoplasmic reticulum (ER)-directed epidermal growth factor (EGF). In other embodiments, the transgenic legume (e.g., a transgenic soybean) produces a soluble, bioactive, endoplasmic reticulum (ER)-directed human epidermal growth factor (hEGF).

In some embodiments, the transgenic soybean produces a soluble, bioactive epidermal growth factor (EGF) protein. In other embodiments, the transgenic soybean produces a soluble, bioactive human epidermal growth factor (hEGF) protein. In some embodiments, the transgenic soybean produces a soluble, bioactive, endoplasmic reticulum (ER)-directed epidermal growth factor (EGF). In some embodiments, the soluble, bioactive, human epidermal growth factor (hEGF) protein is an endoplasmic reticulum (ER)-directed protein.

In some embodiments, the EGF protein is encoded by nucleic acid sequence according to SEQ ID NO: 1 or SEQ ID NO: 26 or a polynucleotide at least 90% identical thereto, wherein the polynucleotide encodes a protein having hEGF activity or a functional fragment thereof. In some embodiments, the nucleotide sequence encodes a protein of SEQ ID NO: 12 or a polynucleotide sequence at least 90% identical thereto encoding a protein having hEGF activity or a functional fragment thereof.

In some embodiments, the protein produced by the transgenic legumes described herein (e.g., a transgenic soybean) is a therapeutic protein, a bioregulatory protein, or an antibody. In other embodiments, the protein produced by the transgenic legumes described herein (e.g., a transgenic soybean) is a growth factor protein. Non-limiting examples of growth factor proteins include but are not limited to epidermal growth factor (EGF), fibroblast growth factor (FGF), platelet-derived growth factor (PDGF), vascular endothelial growth factor (VEGF), insulin-like growth factor (IGF), heat shock proteins (HSP; e.g., HSP 27, HSP 60, HSP 70, HSP 90), transforming growth factor (TGF)-alpha, TGF-beta, tumor necrosis factor (TNF)-alpha, Interleukin (IL)-1, Interferons, connective tissue growth factor (CTGF), hepatocyte scatter factor (HGF), granulocyte macrophage colony stimulating factor (GM-CSF), or a combination thereof. In some embodiments, the bioregulatory or therapeutic protein comprises insulin, fibronectin, or hypoxia-inducible factor (HIF)-1 alpha. In some embodiments, the bioregulatory or therapeutic protein comprises transcription factors or other related proteins.

As used herein, a “therapeutic protein” refers to a protein utilized to induce a stabilization, reparative or regenerative process aimed at inducing a restorative or therapeutic effect. As used herein, a “bioregulatory protein” refers to proteins that regulate biological processes in the body. In some embodiments, “bioregulatory protein” and “therapeutic protein” may be used interchangeably.

In some embodiments, the medical materials described herein may further comprise an anti-inflammatory agent, an antifungal agent, an antibacterial agent, or a combination thereof. In some embodiments, the anti-inflammatory agent comprises steroids, sterols, and nonsteroidal anti-inflammatory agents. In some embodiments, the antifungal agent comprises polyenes (e.g., amphotericin, nystatin); Azoles—Imidazoles (e.g., Clotrimazole, Ketoconazole); Triazoles (e.g., Itraconazole); Allylamines (e.g., butenafine, terbinafine); Echinocandins (e.g., Caspofungin), Griseofulvin, Tolnaftate, or a combination thereof. In some embodiments, the antibacterial agent comprises tetracycline, doxycycline, or other antibiotics.

In some embodiments, the medical material allows for local delivery of the animal or human protein, the anti-inflammatory agent, the antibacterial agent, or a combination thereof. In some embodiments, the medical material described herein can be utilized for external or internal use in a subject.

In some embodiments, the medical material described herein is for epidermal applications. In other embodiments, the medical material described herein is for dermal applications.

In some embodiments, the medical material described herein may be applied internally to a gastrointestinal tract of a subject. The medical material may also be applied to an abscess.

In some embodiments, the medical material described herein further comprises a bioadhesive. In some embodiments, the bioadhesive is disposed on a surface of the medical material.

In some embodiments, the medical material comprises a hygroscopic material.

In some embodiments, the medical material described herein further comprises a non-active plant element, a synthetic element, an excipient, or an adjuvant.

In some embodiments, the non-active plant element or the synthetic element is hygroscopic. In other embodiments, the non-active plant element or the synthetic element is hydrophobic. In further embodiments, the non-active plant element or the synthetic element is swellable. In some embodiments, the non-active plant element or the synthetic element is hydrophilic. In some embodiments, the non-active plant element or the synthetic element comprises hydrogel, aerogel or organogels, or a combination thereof.

In some embodiments, the excipient or the adjuvant comprises a colloidal binder, gelatin, polyethylene glycol (PEG), PEG-lactide, Plutronics, Tetronics, Carbopol, Eudragits, Agar, Pectin, Guar gum, alginates, polyvinyl alcohol (PVA), carboxymethylcellulose, hyaluronic acid, or a combination thereof.

In some embodiments, the medical material described herein further comprises a marker or sensor, or means of detection. In some embodiments, the sensor is for providing feedback information as to the status of the topical condition and subepidermal or subdermal condition under or adjacent to the location in which the said medical material is applied. In some embodiments, the marker is a pH indicator. In other embodiments, the marker is a redox indicator. In some embodiments, the marker is for detecting infection.

In some embodiments, the medical material described herein further comprises a medication. In some embodiments, the medication is an anti-inflammatory agent, an antibacterial agent, an antimicrobial agent, an antifungal agent, an anesthetic agent, or a combination thereof.

Non-limiting examples of anti-inflammatory agents include but are not limited to steroids, sterols, and nonsteroidal anti-inflammatory agents. Non-limiting examples of antibacterial agents include but are not limited to tetracycline, doxycycline, or other antibiotics.

In some embodiments, the medical material described herein further comprises a perfumant. In other embodiments, the medical material described herein comprises a compound for reducing odor.

In some embodiments, the medical material described herein further comprises non-allergenic soy, a cell, or a cell product. In some embodiments, the material comprises or delivers a cell product or constituent such as platelet-rich plasma (prp) or extract, a viral vector, gene, plasmid, episome or bacteriophage, siRNA, aptamer, genetic material, bacteriophage, or a combination thereof. In some embodiments, the cell or cell product delivers live, dead, or attenuated epithelial cells, platelets, or white blood cells, or a combination thereof.

In some embodiments, the present invention features a method of treating a topical condition. In some embodiments, said method comprises applying a medical material to the topical condition described herein. In some embodiments, the medical material reduces inflammation.

In other embodiments, the present invention features a treatment system involving the application of the medical material described herein (i.e., therapeutic construct), monitoring its status via the contained sensors/indicators (e.g., a pH indicator) and removing and/or re-application pending sensor readout.

The present invention features methods and compositions for producing epidermal growth factor (EGF) (e.g., human EGF) (SEQ ID NO: 35) in soybean seeds. For example, the present invention features methods for producing EGF in soybeans seeds, as well as genes for introducing into soybeans to produce EGF, transgenic soybeans engineered to produce EGF, and soymilk compositions comprising soybean-derived EGF.

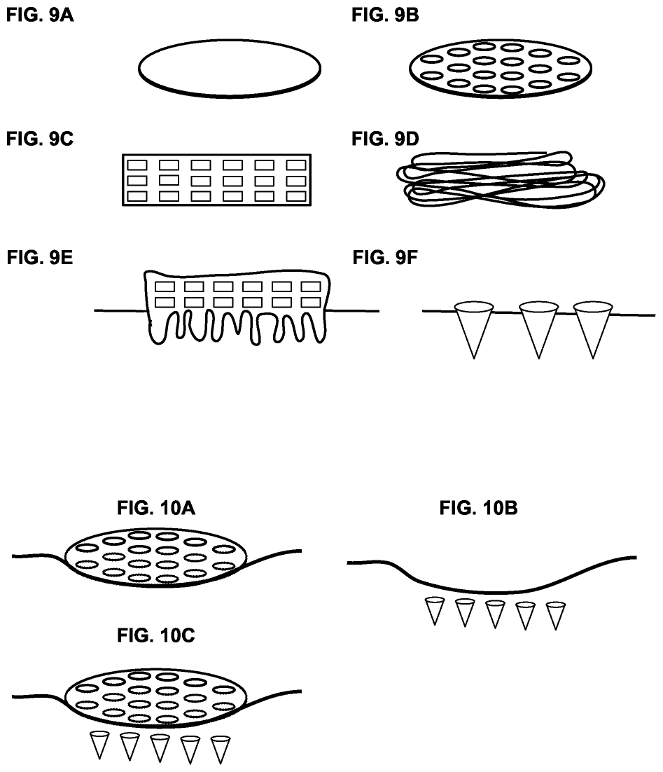

In some embodiments, the medical material described herein comprises a partially vacuous, discontinuous, or holey construct (FIG. 9C). In other embodiments, the medical material described herein is fabricated as a gauze, mesh, sheet, film, fibrous construct, or a bandage.

In some embodiments, the medical material described herein is constructed by spin-coating, drop casting, spin casting, extrusion, electrospinning, film formation spraying, spray drying, drop casting, spin casting, extrusion, electrospinning, low-temperature thermoforming, micro-particle formation, nano-particle formation, micro-capsule formation, nano-capsule formation, or a combination thereof.

In some embodiments, the medical material described herein may form a medical construct (e.g., a sheet, gauze, foam, or bandage for body use). In some embodiments, the medical construction is for epidermal or dermal application.

In some embodiments, the medical construct can be utilized for external or internal use in a subject. In some embodiments, the medical material is applied internally to an endoluminal tract of the subject. In some embodiments, the endoluminal tract comprises a gastrointestinal tract or a respiratory tract of the subject. In some embodiments, the subject is a mammal. In some embodiments, the subject is human.

In some embodiments, the medical construct comprises a partially vacuous, discontinuous, or holey construct. In some embodiments, the medical construct is constructed by spin-coating, drop casting, spin casting, extrusion, electrospinning, film formation spraying, spray drying, drop casting, spin casting, extrusion, electrospinning, low-temperature thermoforming, micro-particle formation, nano-particle formation, micro-capsule formation, nano-capsule formation, or a combination thereof.

In some embodiments, the medical construct is fabricated as a gauze, mesh, sheet, film, fibrous construct, or bandage.

In some embodiments, the gauze is monolithic. In some embodiments, the gauze is dense or porous. In some embodiments, the gauze is about 30 cm×30 cm, or about 20 cm×20 cm, or about 10 cm×10 cm, or about 40 cm×40 cm, or about 50 cm×50 cm. In some embodiments, the gauze acts as a hygroscopic absorbent.

In some embodiments, the medical construct comprises a multi-layered sheet. In some embodiments, the multi-layered sheet comprises an additional hygroscopic material. In some embodiments, the hygroscopic material comprises a swellable, hydrogel material (e.g., PEG, PEG-lactide, Pluronic, Tetronic, Carbomer, or a combination thereof). In some embodiments, the swellable, hydrogel material allows for wound fluid absorption. In some embodiments, the multi-layered sheet comprises an external hydrophobic layer. In some embodiments, the hydrophobic layer comprises polyethylene or perforated polyethylene. The perforated polyethylene may allow air exchange, removal of odor, or a combination thereof.

In some embodiments, the medical construct is a plug. The plug may comprise multiple layers. In some embodiments, the plug can be applied to internal or external wounds.

The present invention may further feature a therapeutic medicament comprising the medical material as described herein. In some embodiments, the therapeutic medicament comprises a cream, an ointment, a salve, or other balms. In some embodiments, the medical material is a powder. In some embodiments, the medical material is combined with an aqueous or non-aqueous base

Non-limiting examples of non-aqueous bases (e.g., organic non-aqueous bases) include but are not limited to ointment bases comprising petrolatum and/or mineral oil, or petrolatum and waxy/fatty alcohol combinations. Non-limiting examples of aqueous bases include but are not limited to guar gum, locust bean gum, quince seed, carrageenan, etc. microbe-based (polysaccharides) Xanthan gum, dextran, hyaluronate, etc. Animal-based (proteins) gelatin, casein, collagen, etc. Cellulose-based Methylcellulose, ethylcellulose, hydroxyethylcellulose, etc. Starch-based Alginic acid-based, Vinyl-based, Other Soluble starch, etc. Alginate, etc. Polyvinyl alcohol, polyvinylpyrrolidone, carboxyvinyl polymer, sodium polyacrylate, etc. Polyethylene glycol.

The present invention shows the accumulation of human EGF (hEGF) in genetically engineered soybean seeds. Further, the present invention shows that the recombinant EGF is indistinguishable from authentic human EGF and is bioactive at stimulating EGF receptor (EGFR) activity. Briefly, the present invention utilizes transgenic soybean seeds expressing a seed-specific codon optimized gene encoding of the human EGF protein with an added ER signal tag at the N terminal. Seven independent lines were grown to be homozygous and found to accumulate a range of 6.7+/−3.1 to 129.0+/−36.7 ug EGF/g of dry soybean seed. Proteomic and immunoblot analysis indicate that the inserted EGF is the same as the human EGF protein. Phosphorylation and immunohistochemical assays on the EGF receptor in HeLa cells indicate the EGF protein produced in soybean seed is bioactive and comparable to commercially available human EGF.

To produce hEGF in soybean, a strong soybean seed-specific promoter, and terminator was used to regulate gene expression of a synthetic soybean codon optimized hEGF (ShEGF) gene that included an N-terminal 60 nucleotide ER-signal sequence (FIG. 1A). In the engineering strategy for the hEGF expression in soybean, the components of the prepro portions of hEGF were eliminated in preference to produce only the final recombinant hEGF product. To facilitate the co-translational transfer of the EGF into the ER lumen for disulfide bond formation, a plant signal sequence was added so that the hEGF synthesized would be as a pre-hEGF. The Gly::ShEGF construct was used for biolistic transformation of soybean somatic embryo cells as outlined in Schmidt M A, Herman E M, Plant Biotechnol J. 2008; 6: 832-842; Schmidt M A, Herman E M, Mol Plant. 2008; 1: 910-924; Schmidt M A, Parrott W A, Hildebrand D F, Berg R H, Cooksey A, Pendarvis K, et al., Plant Biotechnol J. 2015; 13: 590-600; and Schmidt M A, Tucker D M, Cahoon E B, Parrott W A. Plant Cell Rep. 2004; 24: 383-391. Embryos were selected in liquid culture by hygromycin B, and individual regenerated lines were separated, propagated, and induced to form cotyledonary embryos. The cotyledonary embryos were evaluated for hEGF production using EGF-specific ELISA, indicating a variation of heterologous protein production. The most promising EGF-expressing lines were moved forward for regeneration by desiccating and subsequent germination. The initial TO generation EGF transgenic plants were grown in the greenhouse and further selected by genomic PCR for an additional 2-3 generations. Additionally, each generation of seeds produced by the selected lines were assayed for hEGF content by ELISA. The hEGF content of each line in seeds representative of the homozygous population is shown in FIG. 1B. The lines varied in hEGF content, but seeds within each line had a narrow range of hEGF accumulation. The EGF transgenic Line 5 produced in excess of 100 μg hEGF per gm dry seed weight, a level calculated to be much in excess of potential therapeutic requirements. By comparison, yeast stains have been used as an expression system for both human EGF and mouse EGF with the highest levels produced being from a multicopy insert Pichia pastoris clone secreting 49 μg EGF/ml. In both the mouse and human EGF yeast production systems, truncated versions of the EGF were detected.

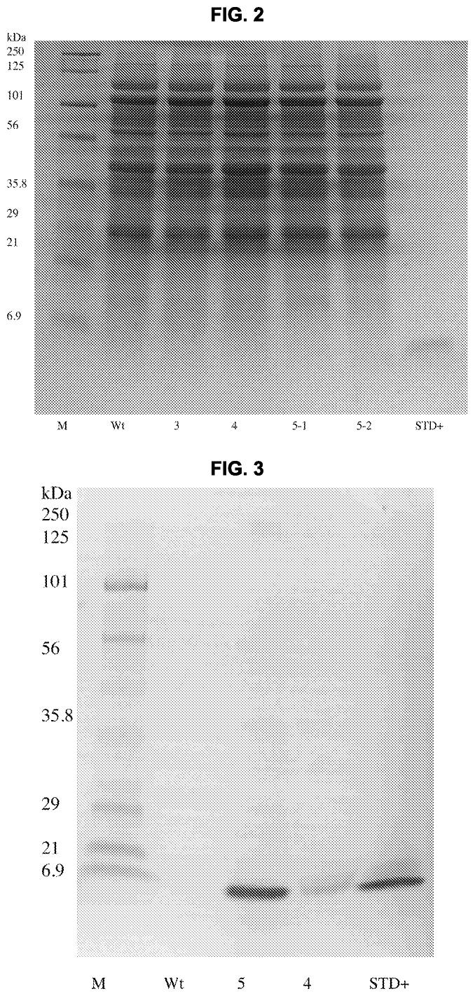

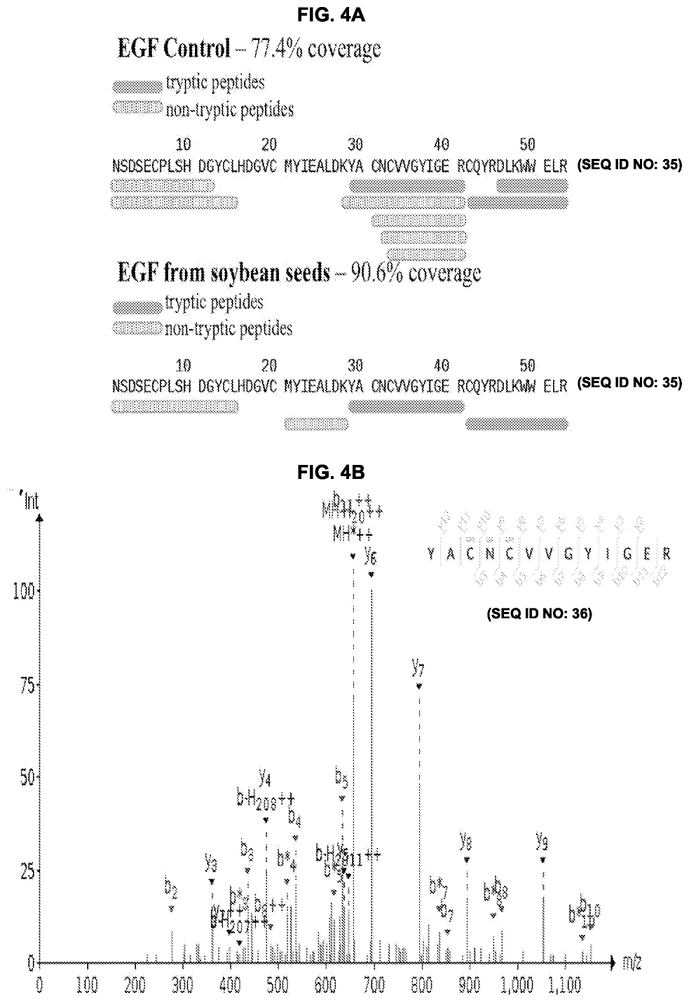

The hEGF soybeans and non-transgenic soybeans were evaluated to determine the biochemical authenticity of the soybean-produced EGF protein. Using 1D SDS/PAGE and parallel immunoblots probed with anti-EGF, the soluble low molecular weight (<10 kDa) seed proteins and the Mr of the soybean-produced hEGF were evaluated. The total protein polypeptide of the hEGF expressing lines appeared to be identical to the standard parental control (See FIG. 2). Immunoblots of the 1D SDS/PAGE probed with anti-EGF showed a lack of an immunoreactive band in the non-transgenic soybean seed control and recognized a 6 kDa Mr band in the hEGF expressing Lines 5 and 4. The soybean-produced hEGF has the same apparent Mr as authentic recombinant hEGF fractioned in an adjacent lane (see FIG. 3). To further assess the soybean-synthesized hEGF, the seed lysates were enriched in low Mr total proteins and concentrated. The crude low Mr proteins were reduced, alkylated, and cleaved with trypsin prior to analysis by mass spectrometry. The resulting data was queried with the hEGF sequence, and exact matches for peptides encompassing the majority of the sequence of the complete, mature hEGF protein were obtained (see FIG. 4). Together, the data shows that transgenic soybeans successfully produced and accumulated hEGF that is, the correct Mr, is immunoreactive with antibodies directed at authentic EGF in both ELISA and immunoblot assay, and that a majority mass spectrometry of fragments of the soybean-produced hEGF match the human EGF sequence.

Soybean-Milk is Compatible with EGF Bioactivity

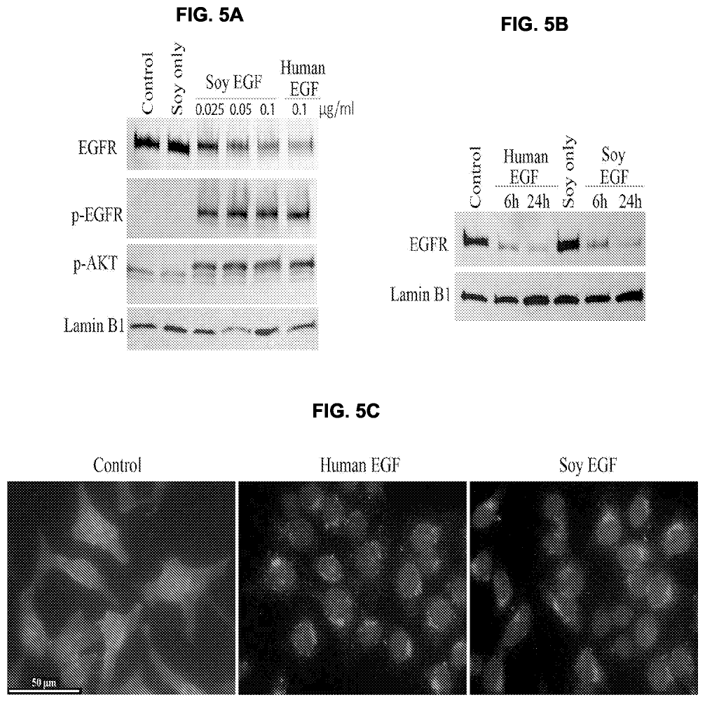

To evaluate the potential of EGF activity in soymilk delivery, commercial recombinant human EGF (rhEGF) was added as a supplement to soymilk and the intrinsic activity of the EGF was tested with a HeLa cell assay. FIG. 5 shows the effects of soymilk on the display of the EGF receptor (EGFR) on Hela cells and the effect of commercial rhEGF supplement to soymilk. Soymilk does not modify the display of EGFR on Hela cells showing that soymilk alone is biologically inactive. The binding of EGF to EGFR results in the decrease of displayed EGFR as it is internalized into the HeLa cells. Hela cells treated with commercially available recombinant rhEGF-supplemented soymilk display the same decrease in EGFR as cells treated with rhEGF in media without soymilk. Parallel time-course experiments show that the effect of rhEGF binding to EFGR is rapid, with a reduction of displayed EGFR occurring within 5 min of treatment and continuing out to at least 30 min. Together these assays show that at this time, soymilk has no apparent negative bioactivity with respect to both the binding of commercial rhEGF to the HeLa cell EGFR or the viability of the HeLa cells over the course of the assay.

Soybean-Synthesized hEGF is Bioactive

To assess the bioactivity of soybean-produced hEGF, samples were prepared from both ShEGF transgenic soybean lines and non-transgenic controls that were used to stimulate HeLa cells to induce EGFR internalization, degradation, and phosphorylation. As shown in FIG. 5, soybean-produced hEGF induces the internalization, degradation, and phosphorylation of EGFR, which is indistinguishable from the bioactivity of commercial rhEGF delivered in control samples. In contrast, samples prepared from control non-transgenic soybeans exhibited no apparent bioactivity showing the degradation and phosphorylation of EGFR is the result of EGF binding of either commercial rhEGF added to the media or from the hEGF produced by the transgenic soybeans. Together these results show that at this time, non-transgenic soybean seeds have no intrinsic EGF-mimic activity able to induce EGFR degradation or phosphorylation, while soybeans producing hEGF have identical activity in comparison to commercial rhEGF.

Synthesis of hEGF does not Affect Overt Soybean Seed Composition

To test for potential collateral composition in the hEGF-producing soybeans, the ShEGF transgenic and non-transgenic control soybeans were analyzed by non-targeted proteomics and metabolomics. Among the significant proteins identified include various well-documented allergens and anti-metabolite proteins. A comparison of standard soybeans with hEGF-producing soybean lines showed that there was no significant difference (p=0.01) between non-transgenic control and ShEGF transgenic soybeans aside from the targeted production of hEGF for any other proteins of concern. This data is available in the PRIDE partner repository with the dataset identifier PXD003326 and 10.6019/PXD003326.

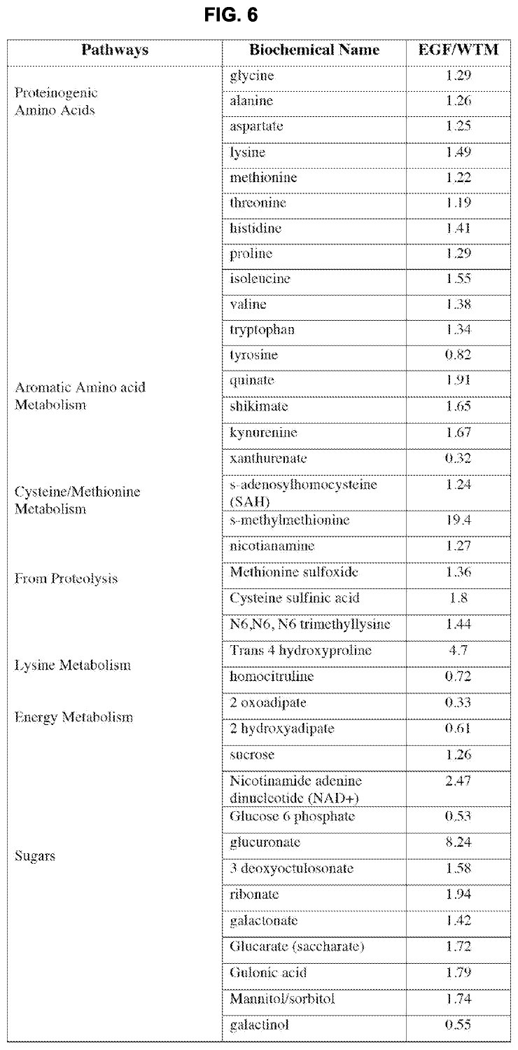

Non-targeted small molecule metabolomics was used to conduct a parallel analysis of the non-transgenic and hEGF soybeans. Again there were insignificant differences between non-transgenic soybean seeds and the ShEGF transgenic seeds (see FIG. 6), with one notable exception. Soybean highly regulates sulfur availability and its allocation into protein. From a nutritional perspective, soybean is considered a somewhat sulfur-deficient crop. There have been a number of biotechnology experiments to increase sulfur content by either modifying assimilation and biosynthesis pathways leading to methionine or over-expressing high-methionine proteins such as Maize zeins. Modifying sulfur by pathway or competition has an effect on sulfur-responsive proteins, including the Bowman-Birk trypsin inhibitor (BBI) and beta chain of the storage protein conglycinin. EGF is a high sulfur content protein that broadly mimics BBI as a small globular protein synthesized by the ER and presumptively competing for sulfur amino acid charge tRNA. Expressing hEGF in soybean has an effect on metabolites involved in sulfur amino acid metabolism that is consistent with producing a protein of EGF's composition. Among the assayed molecules of particular note is the soybean molecule Genistein, an isoflavone that has been shown to affect the activity of tyrosine phosphatase in the signal cascade associated with EGF signaling. Genistein levels were determined to be the same in both the non-transgenic and hEGF-expressing soybean lines. This, too, helps demonstrate that the expression of hEGF in soybeans does not produce any incidental collateral consequences of concern for its potential therapeutic use.

### Example 1

The following Example describes non-limiting methods associated with the present invention.

### Transgenic EGF Soybean Seeds

Epidermal growth factor protein from humans was produced in soybean seeds by constructing a plant gene expression cassette that involved a synthetic codon-optimized EGF nucleotide sequence (protein sequence from Genbank accession CCQ43157). This 162 bp open reading frame was placed in-frame behind a 20-amino acid endoplasmic reticulum (ER) signal sequence from the Arabidopsis chitinase gene. The ER-directed EGF encoding open reading frame was developmentally regulated by the strong seed-specific storage protein glycinin regulatory elements. The entire seed-specific cassette to direct EGF production was placed in a vector containing the hygromycin resistance gene under the strong constitutive expression of the potato ubiquitin 3 regulatory elements as previously described (Schmidt M A, Herman E M. The Collateral Protein Compensation Mechanism Can Be Exploited To Enhance Foreign Protein Accumulation In Soybean Seeds. Plant Biotechnol J. 2008; 6: 832-842; Schmidt M A, Herman E M. An RNAi knockdown of soybean 24 kDa oleosin results in the formation of micro-oil bodies that aggregate to form large complexes of oil bodies and ER-containing caleosin. Mol Plant. 2008; 1: 910-924; Schmidt M A, Parrott W A, Hildebrand D F, Berg R H, Cooksey A, Pendarvis K, et al. Transgenic soybean seeds accumulating β-carotene exhibit the collateral enhancements of high oleate and high protein content traits. Plant Biotechnol J. 2015; 13: 590-600). The result plasmid pGLY::ShEGF was sequenced using a glycinin promoter primer (5′ TCATTCAC CTTCCTCTCTTC 3′) (SEQ ID NO: 42) to ensure the EGF open reading frame was placed correctly between the regulatory elements. Somatic soybean (Glycine max L. Merrill cv Jack (wild type)) embryos were transformed via biolistics using 30 mg/L hygromycin B selection and regenerated as previously described (Schmidt M A, Tucker D M, Cahoon E B, Parrott W A. Towards normalization of soybean somatic embryo maturation. Plant Cell Rep. 2004; 24: 383-391). Embryos from resistant lines were analyzed by genomic PCR to confirm the presence of inserted hygromycin cassette using primers specific to the hygromycin gene (HygF 5′CTCACTATTCCTTTGCCCTC3′ (SEQ ID NO: 43) and HygR 5′CTGACCTATTGCATCT CCCG3′ (SEQ ID NO: 44)), cetyl trimethyl ammonium bromide (CTAB) extraction genomic DNA isolation and the following amplification conditions: 150 ng genomic DNA in 25 μl total reaction containing 200 nM primers and 3 U Taq polymerase (NEB) and the following cycling parameters (initial 95° C. 4 min then 45 cycles of 95° C. 30 s, 55° C. 45 s, 72° C. 90s; followed by a final extension of 72° C. 7 min). Dry seeds from two successive generations of PCR positive plants were analyzed by ELISA for the expression of EGF protein until all 7 lines were confirmed to be homozygous. EGF transgenic soybean plants along with non-transgenic control wild-type cultivar plants, were grown side by side in a greenhouse at 25° C. under 16 h daylight with 1000 μm-2/s.

As previously discussed, the present invention features compositions comprising the nucleic acid sequence, SEQ ID NO: 1 of Table 1 below. The vector of SEQ ID NO: 1 comprises a modified hEGF gene (the sequence within SEQ ID NO: 1 that encodes hEGF is outlined). The optimized hEGF nucleic acid sequence is not limited to SEQ ID NO: 1 and comprises a nucleic acid that encodes a peptide of interest.

In some embodiments, the nucleic acid is at least about 90% identical to SEQ ID NO: 1. In some embodiments, the nucleic acid is at least about 93% identical to SEQ ID NO: 1. In some embodiments, the nucleic acid sequence is at least about 95% identical to SEQ ID NO: 1. In some embodiments, the nucleic acid sequence is at least about 98% identical to SEQ ID NO: 1. In some embodiments, the nucleic acid sequence is at least about 99% identical to SEQ ID NO: 1. Non-limiting examples of such nucleic acid sequences can be found in Table 1 below. For example, SEQ ID NO: 2 and SEQ ID NO: 7 are sequences for a modified hEGF that is about 99% identical to SEQ ID NO: 1. SEQ ID NO: 3 and SEQ ID NO: 8 are sequences for a modified EGF that is about 98% identical to SEQ ID NO: 1; SEQ ID NO: 4 and SEQ ID NO: 9 are sequences for a modified EGF that is about 95% identical to SEQ ID NO: 1 (note that the bold letters in Table 1 are nucleotide substitutions as compared to SEQ ID NO: 1, and the codon underlined).

TABLE 1
Examples of Nucleic Acid Sequence Identity ≥90% to SEQ ID NO: 1

| SEQ | | | % Alignment |
|---|---|---|---|
| ID | | | to SEQ ID |
| NO | Description | Nucleic Acid Sequence | NO: 1 |
| 1 | Optimized EGF sequence for | tctctttcttcagccgaaaattccgatagtgagtgtc | 100 |
| | soybean transformation | cactctcccatgatggctattgtttgcacgacgga | |
| | | gtttgcatgtatattgaagctttggataagtacgcat | |
| | | gtaactgcgttgtgggatatatcggtgaaagatgc | |
| | | caatacagggacctcaaatggtgggagctgag | |
| | | ataa | |
| 2 | Optimized EGF sequence for | tctctttcttcagccgaaacttccgatagtgagtgtc | 99 |
| | soybean transformation with | cactctcccatgatggctattgtttgcacgacgga | |
| | 2 base substitution for 99% | gttcgcatgtatattgaagctttggataagtacgca | |
| | sequence identity to Seq ID 1 | tgtaactgcgttgtgggatatatcggtgaaagatg | |
| | | ccaatacagggacctcaaatggtgggagctga | |
| | | gataa | |
| 3 | Optimized EGF sequence for | tctctttcttcagccgaaaactccgctagtgagtgt | 98 |
| | soybean transformation with | ccactctcccatgatggctattgtttgcacgacgg | |
| | 4 base substitution for 98% | agttcgcatgtatattgaagctttggataagtacgc | |
| | sequence identity to Seq ID 1 | atataactgcgttgtgggatatatcggtgaaagat | |
| | | gccaatacagggacctcaaatggtgggagctg | |
| | | agataa | |
| 4 | Optimized EGF sequence for | tctctttcttcagccgaaaactccgctagtgagtgt | 95 |
| | soybean transformation with | tcactctcccatgatggcgattgtttgcacgacgg | |
| | 9 base substitution for 95% | agttcgcatgtatattgaagctttggataagtacgc | |
| | sequence identity to Seq ID 1 | atataactgcgttgtggaatatatcggtgaaaga | |
| | | ggccaatacagggacctcaaacggtgggagct | |
| | | gagataa | |
| 5 | Optimized EGF sequence for | tctctttcttcagccgaaaactccgctattgagtgtt | 93 |
| | soybean transformation with | cactctcccctgatggcgattgtttgcacgacgga | |
| | 13 base substitution for 93% | gttcgcatgtatattgaagctttgtataagtacgcat | |
| | sequence identity to Seq ID 1 | ataactgcgttgtggaatatatcggtgaaagagg | |
| | | ccaatacaggaacctcaaacggtgggagctga | |
| | | gataa | |
| 6 | Optimized EGF sequence for | tctctttcttcagccgaaaactccgctattgagtgtt | 90 |
| | soybean transformation with | cactctcccctgatggcgattgtttgcaagacgta | |
| | 18 base substitution for 90% | gttcgcatgtatagtgaagctttgtataagtacgc | |
| | sequence identity to Seq ID 1 | atataactgcgttgtggaatatctcggtgaaaga | |
| | | ggccaatacaggaacctcaaacggtggaagct | |
| | | gagataa | |
| 7 | Optimized EGF sequence for | tctctttcttcagccgaaaatcccgatagtgagtgt | 99 |
| | soybean transformation with | ccactctgccatgctggctattgttcgcacgacgg | |
| | 2 base substitution for 99% | agtttgcatgtatattgtagctgtggataagtacgc | |
| | sequence identity to Seq ID 1 | atgtaactgcgctgtgggatatatcggtgcaagat | |
| | | gccaatacagcgacctcaaatggtgggacccg | |
| | | agataa | |
| 8 | Optimized EGF sequence for | tctctttcttcagccgaaaatcccgatcgtgagtgt | 98 |
| | soybean transformation with | ccactctgccatgctggctattgttcgcacgacgg | |
| | 4 base substitution for 98% | agtttgcatgtatattgtagctgtggataagtacgc | |
| | sequence identity to Seq ID 1 | atgtaactgcgctgtgggatatatcggtgcaagat | |
| | | gccaatacagcgacctcaaatggtgggacccg | |
| | | agataa | |
| 9 | Optimized EGF sequence for | tctctttcttcagccgaaaatcccgatcgtgagtgt | 95 |
| | soybean transformation with | ccactctgccatgctggctattgttcgcacgacgg | |
| | 9 base substitution for 95% | agtttgcatgtatattgtagctgtggataagtacgc | |
| | sequence identity to Seq ID 1 | atgtaactgcgctgtgggatatatcggtgcaagat | |
| | | gccaatacagcgacctcaaatggtgggacccg | |
| | | agataa | |
| 10 | Optimized EGF sequence for | tctctttcttcagccgaaaatcccgatcgtgcgtgt | 93 |
| | soybean transformation with | ccactctgccatgctgtctattgttcgcacgacgg | |
| | 13 base substitution for 93% | agtttgcatgtatattgtagctgtggataagtacgc | |
| | sequence identity to Seq ID 1 | atgtaactgcgctgtgggatatatcggtgcaagat | |
| | | gccaatacagcgacctcaaatggtgggacccg | |
| | | agataa | |
| 11 | Optimized EGF sequence for | tctctttcttcagccgaaaatcccgatcgtgcgtgt | 90 |
| | soybean transformation with | ctactctgccatgctgtctattgttcgcacgacag | |
| | 18 base substitution for 90% | agtttgcatgtatattgtagctgtggataagtactc | |
| | sequence identity to Seq ID 1 | atgtaactgcgctgtgggatgtatcggtgcaaga | |
| | | tgccaatacagcgacctcaattggtgggagccg | |
| | | agataa | |

Bold letters are nucleotide substitutions within a codon; the respective codon is underlined

The vector comprises a nucleic acid that encodes a peptide of interest. In some embodiments, the nucleic acid sequence is at least about 90% identical to SEQ ID NO: 1. In some embodiments, the nucleic acid sequence is at least about 93% identical to SEQ ID NO: 1. In some embodiments, the nucleic acid sequence is at least about 95% identical to SEQ ID NO: 1. In some embodiments, the nucleic acid sequence is at least about 98% identical to SEQ ID NO: 1. In some embodiments, the nucleic acid sequence is at least about 99% identical to SEQ ID NO: 1. Non-limiting examples of resulting amino acid sequences encoded by such nucleic acid sequences can be found in Table 2 below. For example, SEQ ID NO: 12 and SEQ ID NO: 18 are amino acid sequences encoded by modified hEGF polynucleotide sequences of Seq ID NO: 2 and SEQ ID NO: 6, respectively, that are about 99% identical to SEQ ID NO: 1 (note that the bold letters in Table 2 are amino acid substitutions as compared to SEQ ID NO: 12).

TABLE 2
Examples of Amino Acid Sequence with Nucleic Acid Identity ≥90%

| SEQ | | | % Alignment |
|---|---|---|---|
| ID | | | to SEQ ID |
| NO | Description | Amino Acid Sequence | NO: 1 |
| 12 | Optimized EGF sequence for | SLSSAENSDSECPLSHDGYCLHDGV | 100 |
| | soybean transformation | CMYIEALDKYACNCVVGYIGERCQY | |
| | | RDLKWWELR | |
| 13 | Optimized EGF sequence for | SLSSAETSDSECPLSHDGYCLHDGV | 99 |
| | soybean transformation with 2 | RMYIEALDKYACNCVVGYIGERCQY | |
| | base substitution for 99% | RDLKWWELR | |
| | sequence identity to Seq ID 1 | | |
| 14 | Optimized EGF sequence for | SLSSAETSASECPLSHDGYCLHDGV | 98 |
| | soybean transformation with 4 | RMYIEALDKYAYNCVVGYIGERCQY | |
| | base substitution for 98% | RDLKWWELR | |
| | sequence identity to Seq ID 1 | | |
| 15 | Optimized EGF sequence for | SLSSAETSASECSLSHDGDCLHDGV | 95 |
| | soybean transformation with 9 | RMYIEALDKYAYNCVVEYIGERGQY | |
| | base substitution for 95% | RDLKRWELR | |
| | sequence identity to Seq ID 1 | | |
| 16 | Optimized EGF sequence for | SLSSAETSAIECSLSPDGDCLHDGV | 93 |
| | soybean transformation with | RMYIEALYKYAYNCVVEYIGERGQY | |
| | 13 base substitution for 93% | RNLKRWELR | |
| | sequence identity to Seq ID 1 | | |
| 17 | Optimized EGF sequence for | SLSSAETSAIECSLSPDGDCLQDVV | 90 |
| | soybean transformation with | RMYSEALYKYAYNCVVEYLGERGQ | |
| | 18 base substitution for 90% | YRNLKRWKLR | |
| | sequence identity to Seq ID 1 | | |
| 18 | Optimized EGF sequence for | SLSSAENADSECPLSHDGYCLHDG | 99 |
| | soybean transformation with 2 | VCMYIVALDKYACNCVVGYIGERCQ | |
| | base substitution for 99% | YRDLKWWELR | |
| | sequence identity to Seq ID 1 | | |
| 19 | Optimized EGF sequence for | SLSSAENADRECPLSHAGYCLHDG | 98 |
| | soybean transformation with 4 | VCMYIVALDKYACNCVVGYIGERCQ | |
| | base substitution for 98% | YRDLKWWELR | |
| | sequence identity to Seq ID 1 | | |
| 20 | Optimized EGF sequence for | SLSSAENADRECPLCHAGYCSHDG | 95 |
| | soybean transformation with 9 | VCMYIVALDKYACNCAVGYIGERCQ | |
| | base substitution for 95% | YSDLKWWEPR | |
| | sequence identity to Seq ID 1 | | |
| 21 | Optimized EGF sequence for | SLSSAENADRACPLCHAVYCSHDG | 93 |
| | soybean transformation with | VCMYIVAVDKYACNCAVGYIGARCQ | |
| | 13 base substitution for 93% | YSDLKWWEPR | |
| | sequence identity to Seq ID 1 | | |
| 22 | Optimized EGF sequence for | SLSSAENADRACLLCHAVYCSHDR | 90 |
| | soybean transformation with | VCMYIVAVDKYSCNCAVGCIGARCQ | |
| | 18 base substitution for 93% | YSDLNWWEPR | |
| | sequence identity to Seq ID 1 | | |

Bold letters are nucleotide substitutions within a codon; the respective codon is underlined.

The present invention also features compositions comprising nucleic acid SEQ ID NO: 26 of Table 3 below. The vector of SEQ ID NO: 1 comprises a modified hEGF gene comprising a modified polynucleotide for the protein-coding region of hEGF, SEQ ID NO: 26 (the sequence within SEQ ID NO: 1 that encodes hEGF is outlined). The optimized hEGF nucleic acid protein-coding sequence is not limited to SEQ ID NO: 26 and comprises a nucleic acid that encodes a peptide of interest.

In some embodiments, the hEGF protein-coding nucleotide sequence is at least 90% identical to SEQ ID NO: 26. In some embodiments, the nucleic acid is at least 93% identical to SEQ ID NO: 26. In some embodiments, the nucleic acid is at least 95% identical to SEQ ID NO: 26. In some embodiments, the nucleic acid is at least 98% identical to SEQ ID NO: 26. In some embodiments, the nucleic acid is at least 99% identical to SEQ ID NO: 26. Non-limiting examples of such nucleic acid sequences can be found in Table 3 below. For example, SEQ ID NO: 27 is a sequence for a modified hEGF that is about 99% identical to SEQ ID NO: 26. SEQ ID NO: 28 is a sequence for a modified EGF that is about 98% identical to SEQ ID NO: 26; SEQ ID NO: 29 is a sequence for a modified EGF that is about 95% identical to SEQ ID NO: 26 (note that the bold letters in Table 3 are nucleotide substitutions as compared to SEQ ID NO: 26, and the codon underlined).

TABLE 3
Examples of Nucleic Acid Sequence Identity ≥90% to Coding Region of SEQ ID NO:

| SEQ | | | % Alignment |
|---|---|---|---|
| ID | | | to SEQ ID |
| NO | Description | Nucleic Acid Sequence | NO: 26 |
| 26 | Coding Region of SEQ ID: 1 | aattccgatagtgagtgtccactctcccatgatgg | 100 |
| | | ctattgtttgcacgacggagtttgcatgtatattgaa | |
| | | gctttggataagtacgcatgtaactgcgttgtggg | |
| | | atatatcggtgaaagatgccaatacagggacct | |
| | | caaatggtgggagctgagataa | |
| 27 | Coding Region of SEQ ID: 1 | acttccgatagtgagtgtccactctcccatgatgg | 99 |
| | with 2 base substitution for | ctattgtttgcacgacggagttcgcatgtatattga | |
| | 99% sequence identity to Seq | agctttggataagtacgcatgtaactgcgttgtgg | |
| | ID 26 | gatatatcggtgaaagatgccaatacagggacc | |
| | | tcaaatggtgggagctgagataa | |
| 28 | Coding Region of SEQ ID: 1 | aactccgctagtgagtgtccactctcccatgatgg | 98 |
| | with 4 base substitution for | ctattgtttgcacgacggagttcgcatgtatattga | |
| | 98% sequence identity to Seq | agctttggataagtacgcatataactgcgttgtgg | |
| | ID 26 | gatatatcggtgaaagatgccaatacagggacc | |
| | | tcaaatggtgggagctgagataa | |
| 29 | Coding Region of SEQ ID: 1 | aactccgctagtgagtgttcactctcccatgatgg | 95 |
| | with 9 base substitution for | cgattgtttgcacgacggagttcgcatgtatattga | |
| | 95% sequence identity to Seq | agctttggataagtacgcatataactgcgttgtgg | |
| | ID 26 | aatatatcggtgaaagaggccaatacagggac | |
| | | ctcaaacggtgggagctgagataa | |
| 30 | Coding Region of SEQ ID: 1 | aactccgctattgagtgttcactctcccctgatgg | 93 |
| | with 12 base substitution for | cgattgtttgcacgacggagttcgcatgtatattga | |
| | 93% sequence identity to Seq | agctttgtataagtacgcatataactgcgttgtgga | |
| | ID 26 | atatatcggtgaaagaggccaatacaggaacct | |
| | | caaacggtgggagctgagataa | |
| 31 | Coding Region of SEQ ID: 1 | aactccgctattgagtgttcactctcccctgatgg | 90 |
| | with 17 base substitution for | cgattgtttgcaagacgtagttcgcatgtatagtg | |
| | 90% sequence identity to Seq | aagctttgtataagtacgcatataactgcgttgtgg | |
| | ID 26 | aatatctcggtgaaagaggccaatacaggaac | |
| | | ctcaaacggtggaagctgagataa | |

Bold letters are nucleotide substitutions within a codon; the respective codon is underlined

The present invention also features compositions comprising nucleic acid sequence, SEQ ID NO: 32 of Table 4 below. The vector of SEQ ID NO: 1 comprises a modified hEGF gene comprising a polynucleotide for the non-hEGF protein coding region, SEQ ID NO: 32. The non-hEGF protein coding sequence of the optimized hEGF nucleotide is not limited to SEQ ID NO: 32. In some embodiments, the 3′ end of SEQ ID NO: 32 is operatively coupled to the 5′ end of SEQ ID NO: 26.

In some embodiments, the non-hEGF protein-coding nucleotide sequence is at least 90% identical to SEQ ID NO: 32. Non-limiting examples of such nucleic acid sequences can be found in Table 4 below. For example, SEQ ID NO: 33 is a sequence that is at least 90% (<100%) identical to SEQ ID NO: 32 (note that the bold letters in Table 4 are nucleotide substitutions as compared to SEQ ID NO: 26, and the codon underlined).

TABLE 4
Examples of Nucleic Acid Sequence
Identity ≥90% to Non-hEGF Protein
Coding Region of SEQ ID NO: 32

| | | | % |
|---|---|---|---|
| SEQ | | | Alignment |
| ID | | Nucleic Acid | to SEQ ID |
| NO | Description | Sequence | NO: 32 |
| 32 | Optimized non-hEGF | tctctttctt | 100 |
| | protein coding region | cagccgaa | |
| | nucleic acid sequence | | |
| 33 | Optimized non-hEGF | tctttttctt | ≥95 |
| | protein coding region | cagccgaa | <100 |
| | sequence with 1 base | | |
| | substitution for at | | |
| | least 90% sequence | | |
| | identity to Seq ID 32 | | |
| 34 | Optimized non-hEGF | tctttttctt | ≥90 |
| | protein coding region | aagccgaa | <95 |
| | sequence with 2 base | | |
| | substitution for at | | |
| | least 90% sequence | | |
| | identity to Seq ID 32 | | |

Bold letters are nucleotide substitutions within a codon; the respective codon is underlined

### EGF Detection Via Immunoblot

Total soluble protein was extracted from dry seeds of two homozygous EGF lines and a non-transgenic control by repeated acetone washes followed by acetone precipitation with the protein pellet dissolved in water. Proteins with molecular weight 10 kDa and under were isolated by separately passing each extract through an Amicon Ultra centrifugal filter (Merck, Kenil-worth NJ). The samples were each suspended in sample buffer (50 mM Tris HCL, pH6.8 2% SDS (w/v), 0.7 M β-mercaptoethanol, 0.1% (w/v) bromphenol blue and 10% (v/v) glycerol) and then denatured 5 min 95° C. Protein content was determined by Bradford assay. A 15% SDS-PAGE gel was used to separate 30 μg protein for each of the three samples: negative control wild type, Lines 4 and 5 of EGF transgenic soybean dry seeds. Commercially available human EGF (Gibco, Life Technologies, United Kingdom) was used at 0.5 μg as a positive control. Gel was electroblotted onto Immobilon P transfer membrane (Millipore, Bedford MA) and blocked with 3% milk solution in TBS for at least 1 hr. The primary antibody was a commercially available anti-EGF (Calbiochem, San Diego CA) and was used in a 1:100 ratio in 3% BSA-TBS buffer overnight at room temperature. After 3 washes of 15 mins each with TBS buffer, the blot was incubated with a 1:10,000 ratio in TBS of secondary antibody anti-rabbit IgG Fab Specific alkaline phosphatase conjugate (Sigma, St. Louis MO). After 3 washes, the presence of the EGF protein was detected by using a color substrate (BCIP/NBT: final concentrations 0.02% (w/v) 5-bromo-4-chloro-3-indolyl phosphate and 0.03% (w/v) nitro blue tetrazolium in 70% (v/v) dimethylformamide) (KPL, Gaithersburg MA).

### EGF Quantification

Total soluble protein was extracted from dry soybean seeds as described previously (Schmidt M A, Herman E M. The Collateral Protein Compensation Mechanism Can Be Exploited To Enhance Foreign Protein Accumulation In Soybean Seeds. Plant Biotechnol J. 2008; 6: 832-842; Schmidt M A, Herman E M. An RNAi knockdown of soybean 24 kDa oleosin results in the formation of micro-oil bodies that aggregate to form large complexes of oil bodies and ER-containing caleosin. Mol Plant. 2008; 1: 910-924) from all 7 lines of pGLY::ShEGF transgenic plants along with non-transgenic seeds as a negative control. EGF was quantitated by commercially available human EGF ELISA assay (Quantikine ELISA kit from R&D systems, Minneapolis MN) according to the manufacturer's instructions. The provided positive control was used to create a standard curve in order to determine the amount of EGF in each soybean protein extract. Each homozygote EGF transgenic line was assayed with three biological replicates, and results were displayed as mean+/− standard error.

### Seed Proteome Composition Analysis

Total soluble proteins were extracted, quantitated, and suspended in a sample loading buffer as previously described (Schmidt M A, Herman E M. The Collateral Protein Compensation Mechanism Can Be Exploited To Enhance Foreign Protein Accumulation In Soybean Seeds. Plant Biotechnol J. 2008; 6: 832-842; Schmidt M A, Herman E M. An RNAi knockdown of soybean 24 kDa oleosin results in the formation of micro-oil bodies that aggregate to form large complexes of oil bodies and ER-containing caleosin. Mol Plant. 2008; 1: 910-924). Approximately 30 μg of protein extract from dry seeds of 4 homozygous EGF lines were separated on a 4-20% gradient SDS-PAGE gel (BioRad, Hercules CA) along with an extract from a non-transgenic seed. The gel was subsequently stained with 0.1% (w/v) Coomassie Brilliant Blue R250 in 40% (v/v) methanol, 10% (v/v) acetic acid overnight and then destained for approximately 3 hrs in 40% methanol, 10% acetic acid with frequent solution changes.

### Mass Spectrometry Analysis to Detect EGF in Soybean Samples

Total soluble protein was extracted from 3 biological EGF transgenic soybean dry seed samples, lines 4, 5, and 6. As described above, proteins with molecular weights lower than 10 kDa were concentrated using an Amicon Ultra centrifugal filter (Merck, Kenilworth NJ). Non-transgenic seeds were used as a negative control, and 5 μg commercially available EGF (as above in the immunoblot section) was a positive control. Protein was precipitated by adjusting the solution to 20% (v/v) trichloroacetic acid and allowed to sit at 4° C. overnight. Precipitated proteins were pelleted using centrifugation, washed twice with acetone, and dried using vacuum centrifugation. The commercial EGF was not filtered or precipitated, only dried. Dried pellets were rehydrated with the addition of 10 μl 100 mM dithiothreitol in 100 mM ammonium bicarbonate and placed at 85° C. for 5 minutes to reduce disulfide bonds. Samples were then alkylated with the addition of 10 μl iodoacetamide in 100 mM ammonium bromide and placed at room temperature in the dark for 30 minutes. Two μg trypsin in 200 μl 100 mM ammonium bromide was added to each sample and placed at 37° C. overnight for enzymatic digestion. Post trypsin digest samples were desalted using a peptide reverse phase microtrap (Michrom BioResources, Auburn CA), dried, and ultimately resuspended in 2 μl of 2% (v/v) acetonitrile, 0.1% (v/v) formic acid. Separation of peptides was performed using a Dionex U3000 splitless nanoflow HPLC system operated at 333 ml minute using a gradient from 2-50% acetonitrile over 60 minutes, followed by a 15-minute wash with 95% acetonitrile and a 15-minute equilibration with 2% acetonitrile. The C18 column, an in-house prepared 75 μm by 15 cm reverse phase column packed with Halo 2.7 μm, 90 Å C18 material (MAC-MOD Analytical, Chadds Ford PA), was located in the ion source just before a silica emitter. A potential of 2100 volts was applied using a liquid junction between the column and the emitter. A Thermo LTQ Velos Pro mass spectrometer using a nanospray Flex ion source was used to analyze the eluate from the U3000. Scan parameters for the LTQ Velos Pro were one MS scan followed by 10 MS/MS scans of the 5 most intense peaks. MS/MS scans were performed in pairs, a CID fragmentation scan followed by an HCD fragmentation scan of the same precursor m/z. Dynamic exclusion was enabled with a mass exclusion time of 3 min and a repeat count of 1 within 30 sec of the initial m/z measurement. Spectra were collected over the entirety of each 90-minute chromatography run. Raw mass spectra were converted to MGF format using MSConvert, part of the ProteoWizard software library (Kessner D, Chambers M, Burke R, Agus D, Mallick P. ProteoWizard: open source software for rapid proteomics tools development. Bioinformatics. 2008; 24: 2534-2536) X!tandem 2013.09.01.1 (Craig R, Beavis R C. TANDEM: matching proteins with tandem mass spectra. Bioinformatics. 2004; 20: 1466-1467) and OMSSA (Geer L Y, Markey S P, Kowalak J A, Wagner L, Xu M, Maynard D M, et al. Open mass spectrometry search algorithm. J Proteome Res. 2004; 3: 958-964) algorithms were employed via the University of Arizona High Performance Computing Center to perform spectrum matching. Precursor and fragment mass tolerance were set to 0.2 Daltons for both OMSSA and X!tandem. Trypsin cleavage rules were used for both algorithms with up to 2 missed cleavages. Amino acid modifications search consisted of single and double oxidation of methionine, oxidation of proline, N-terminal acetylation, carbamidomethylation of cysteine, deamidation of asparagine and glutamine and phosphorylation of serine, threonine, and tyrosine. X!tandem xml and OMSSA xml results were filtered using Perl to remove any peptide matches with an E-value>0.05 as well as proteins identified by a single peptide sequence. The protein fasta database for Glycine max was downloaded on Aug. 5, 2015, from NCBI RefSeq with the addition of the EGF amino acid sequence. A randomized version of the Glycine max fasta was concatenated to the original as a way to assess dataset quality. The mass spectrometry proteo-mics data have been deposited to the ProteomeXchange Consortium (http://proteomecentral. proteomexchange.org) via the PRIDE partner repository (Guo J, Longshore S, Nair R, Warner B W. Retinoblastoma protein (pRb), but not p107 or p130, is required for the maintenance of enterocyte quiescence and differentiation in the small intestine. J Biol Chem. 2009; 284:134-40) with the dataset identifier PXD003326 and 10.6019/PXD003326.

### Cell Culture, Western Blotting, and Immunocytochemistry

Hela cells (obtained from American Tissue Culture Collection) were cultured in Minimum Essential Media (MEM) complemented with 10% Fetal Bovine Serum (FBS), 100 units/ml penicillin, and 100 μg/ml streptomycin. For the western blotting assay, cells grown in a 6-well plate were kept in serum-free MEM media for 24 hours. Cells were then either kept in a serum-free medium (control) or stimulated with soy milk alone, soy EGF, or commercial recombinant human EGF for different time periods as indicated. Cells were lysed by directly adding 1×SDS sample buffer (50 mM Tris-HCl, pH 6.8, 10% glycerol, 2% SDS, and 5% β-ME) to the cells after washing 3 times with 1×PBS. EGF bio-activity was determined via EGFR phosphorylation and downstream AKT phosphorylation. Total EGFR was also measured since EGFR is known to undergo internalization when stimulated with EGF. Antibodies used in western blot are anti-p-EGFR (Tyr1068) (#2234, Cell Signaling Technology), anti-total EGFR (#06-847, Millipore), anti-p-AKT (#4060, Cell Signaling Technology) and anti-Lamin B1 (#13435, Cell Signaling Technology) [40]. For the immunocytochemistry assay, cells were grown on a coverslip in a 6-well plate and kept in serum-free media for 24 hours before stimulation, cells were then either kept in serum-free media (control) or stimulated with human or soy EGF for 6 hours. Cells were washed with PBS and fixed with 4% formalin. EGFR was labeled using an anti-EGFR antibody (#4267, Cell Signaling Technology) and detected with Alexa Fluor 594 Goat anti-rabbit IgG (#A11012, Life Technologies). The cell nuclei were shown using a mounting medium with DAPI (#H-1200, Vectorshield).

### Embodiments

The following embodiments are intended to be illustrative only and not to be limiting in any way.

Embodiment 1: A medical material comprising at least one soybean protein derived from a transgenic soybean or at least a portion of the transgenic soybean.

Embodiment 2: The medical material of embodiment 1, wherein the at least one soybean protein comprises 7S, 11S, or a combination thereof.

Embodiment 3: The medical material of embodiment 1 or embodiment 2 further comprising an animal or human protein derived from the transgenic soybean or at least a portion of the transgenic soybean, wherein the transgenic soybean produces said animal or human protein.

Embodiment 4: A medical material comprising an animal or human protein derived from a transgenic soybean or at least a portion of the transgenic soybean, wherein the transgenic soybean produces said animal or human protein.

Embodiment 5: The medical material of embodiment 4 further comprising at least one soybean protein derived from a transgenic soybean or at least a portion of the transgenic soybean.

Embodiment 6: The medical material of embodiment 5, wherein the at least one soybean protein comprises 7S, 11S, or a combination thereof.

Embodiment 7: A medical material comprising at least one soybean protein and an animal or human protein derived from a transgenic soybean or at least a portion of the transgenic soybean, wherein the transgenic soybean produces said animal or human protein.

Embodiment 8: The medical material of embodiment 7, wherein the at least one soybean protein comprises 7S, 11S, or a combination thereof.

Embodiment 9: The medical material of any one of embodiments 1-8, wherein the animal or human protein is a growth factor, wherein the growth factor includes epidermal growth factor (EGF), fibroblast growth factor (FGF), platelet-derived growth factor (PDGF), vascular endothelial growth factor (VEGF), insulin-like growth factor (IGF), heat shock proteins (HSP), transforming growth factor (TGF)-alpha, TGF-beta, tumor necrosis factor (TNF)-alpha, Interleukin (IL)-1, Interferons, connective tissue growth factor (CTGF), hepatocyte scatter factor (HGF), granulocyte macrophage colony stimulating factor (GM-CSF), or a combination thereof.

Embodiment 10: The medical material of embodiment 9, wherein the EGF protein comprises a soluble, bioactive, human epidermal growth factor (hEGF) protein.

Embodiment 11: The medical material of embodiment 10, wherein the soluble, bioactive, human epidermal growth factor (hEGF) protein is an endoplasmic reticulum (ER)-directed protein.

Embodiment 12: The medical material of embodiment 11, wherein the EGF protein is encoded by nucleic acid sequence according to SEQ ID NO: 1 or SEQ ID NO: 26 or a polynucleotide at least 90% identical thereto, wherein the polynucleotide encodes a protein having hEGF activity, or a functional fragment thereof.

Embodiment 13: The medical material of embodiment 12, wherein the nucleotide sequence encodes a protein of SEQ ID NO: 12 or a polynucleotide sequence at least 90% identical thereto encoding a protein having hEGF activity or a functional fragment thereof.

Embodiment 14: The medical material of any one of embodiments 1-13, wherein the animal or human protein is a therapeutic protein, a bioregulatory protein, or an antibody.

Embodiment 15: The medical material of embodiment 14, wherein the bioregulatory or therapeutic protein comprises insulin, fibronectin, or HIF-1 alpha.

Embodiment 16: The medical material of any one of embodiments 1-15 further comprising an anti-inflammatory agent, wherein the anti-inflammatory agent comprises steroids, sterols, and nonsteroidal anti-inflammatory agents.

Embodiment 17: The medical material of any one of embodiments 1-16, further comprising an antibacterial agent or an antifungal agent; wherein the antibacterial agent comprises tetracycline, doxycycline, or other antibiotics; wherein the antifungal agent comprises polyenes, Azoles—Imidazoles, Triazoles, Allylamines, Echinocandins, Griseofulvin, or Tolnaftate.

Embodiment 18: The medical material of any one of embodiments 1-17, wherein the medical material allows for local delivery of the animal or human protein, the anti-inflammatory agent, the antibacterial agent, or a combination thereof.

Embodiment 19: The medical material of any one of embodiments 1-18 further comprising a bioadhesive, wherein the bioadhesive is disposed on a surface of the medical material.

Embodiment 20: The medical material of any one of embodiments 1-19, wherein the medical material comprises a hygroscopic material.

Embodiment 21: The medical material of any one of embodiments 1-20. wherein the medical material forms a medical construct.

Embodiment 22: A medical construct fabricated from the medical material according to any one of embodiments 1-21.

Embodiment 23: The medical construct of embodiment 22, wherein the construct is for epidermal or dermal application.

Embodiment 24: The medical construct of embodiment 22 or embodiment 23, wherein the medical construct can be utilized for external or internal use in a subject.

Embodiment 25: The medical construct of embodiment 24, wherein the medical material is applied internally to an endoluminal tract of the subject, wherein the endoluminal tract comprises a gastrointestinal tract or a respiratory tract of the subject.

Embodiment 26: The medical material of embodiment 25, wherein the subject is a mammal.

Embodiment 27: The medical material of embodiment 25, wherein the subject is a human.

Embodiment 28: The medical construct of any one of embodiments 22-27, wherein the material comprises a partially vacuous, discontinuous, or holey construct.

Embodiment 29: The medical construct of any one of embodiments 22-28, wherein the medical construct is constructed by spin-coating, drop casting, spin casting, extrusion, electrospinning, film formation spraying, spray drying, drop casting, spin casting, extrusion, electrospinning, low-temperature thermoforming, micro-particle formation, nano-particle formation, micro-capsule formation, nano-capsule formation, or a combination thereof.

Embodiment 30: The medical construct of any one of embodiments 22-29, wherein the medical construct is fabricated as a gauze, mesh, sheet, film, fibrous construct, or bandage.

Embodiment 31: The medical construct of embodiment 30, wherein the gauze is a monolithic gauze.

Embodiment 32: The medical construct of embodiment 30 or embodiment 31, wherein the gauze is dense or porous.

Embodiment 33: The medical construct of any one of embodiments 30-32, wherein the gauze is 30 cm×30 cm.

Embodiment 34: The medical construct of any one of embodiments 30-33, wherein the gauze acts as a hygroscopic absorbent.

Embodiment 35: The medical construct of any one of embodiments 22-29, wherein the medical construct comprises a multi-layered sheet.

Embodiment 36: The medical construct of embodiment 35, wherein the multi-layered sheet comprises an additional hygroscopic material, wherein the hygroscopic material comprises a swellable, hydrogel material.

Embodiment 37: The medical construct of embodiment 36, wherein the swellable, hydrogel material allows for wound fluid absorption.

Embodiment 38: The medical construct of any one of embodiments 35-37, wherein the multi-layered sheet comprises an external hydrophobic layer, wherein the hydrophobic layer comprises polyethylene or perforated polyethylene.

Embodiment 39: The medical construct of embodiment 38, wherein the perforated polyethylene allows air exchange, removal of odor, or a combination thereof.

Embodiment 40: The medical construct of any one of embodiments 22-29, wherein the medical construct is a plug.

Embodiment 41: The medical construct of embodiment 40, wherein the plug comprises multiple layers.

Embodiment 42: The medical construct of embodiment 40 and embodiment 41, wherein the plug can be applied to internal or external wounds.

Embodiment 43: A therapeutic medicament comprising the medical material according to any one of embodiments 1-21.

Embodiment 44: The therapeutic medicament of embodiment 43, wherein the therapeutic medicament comprises a cream, an ointment, a salve, or other balms.

Embodiment 45: The therapeutic medicament of embodiment 43 or embodiment 44, wherein the medical material is a powder.

Embodiment 46: The therapeutic medicament of any one of embodiment 43-45, wherein the medical material is combined with an aqueous or non-aqueous base.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and only for ease of review by the patent office and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of,” and as such, the written description requirement for claiming one or more embodiments of the present invention using the phrase “consisting of” is met.

What is claimed is:

1. A medical material comprising an animal or human protein derived from a transgenic soybean or at least a portion of the transgenic soybean, wherein the transgenic soybean produces said animal or human protein.

2. The medical material of claim 1 further comprising at least one soybean protein derived from a transgenic soybean or at least a portion of the transgenic soybean, wherein the at least one soybean protein comprises 7S, 11S, or a combination thereof.

3. The medical material claim 1, wherein the animal or human protein is a growth factor, wherein the growth factor includes epidermal growth factor (EGF), fibroblast growth factor (FGF), platelet-derived growth factor (PDGF), vascular endothelial growth factor (VEGF), insulin-like growth factor (IGF), heat shock proteins (HSP), transforming growth factor (TGF)-alpha, TGF-beta, tumor necrosis factor (TNF)-alpha, Interleukin (IL)-1, Interferons, connective tissue growth factor (CTGF), hepatocyte scatter factor (HGF), granulocyte macrophage colony stimulating factor (GM-CSF), or a combination thereof.

4. The medical material of claim 3, wherein the EGF protein comprises a soluble, bioactive, human epidermal growth factor (hEGF) protein.

5. The medical material of claim 4, wherein the soluble, bioactive, human epidermal growth factor (hEGF) protein is an endoplasmic reticulum (ER)-directed protein.

6. The medical material of claim 5, wherein the EGF protein is encoded by nucleic acid sequence according to SEQ ID NO: 1 or SEQ ID NO: 26 or a polynucleotide at least 90% identical thereto, wherein the polynucleotide encodes a protein having hEGF activity, or a functional fragment thereof.

7. The medical material of claim 6, wherein the nucleotide sequence encodes a protein of SEQ ID NO: 12 or a polynucleotide sequence at least 90% identical thereto encoding a protein having hEGF activity or a functional fragment thereof.

8. The medical material of claim 1, wherein the animal or human protein is a therapeutic protein, a bioregulatory protein, or an antibody.

9. The medical material of claim 8, wherein the bioregulatory or therapeutic protein comprises insulin, fibronectin, or HIF-1 alpha.

10. The medical material of claim 1, further comprising an anti-inflammatory agent, wherein the anti-inflammatory agent comprises steroids, sterols, and nonsteroidal anti-inflammatory agents.

11. The medical material of claim 1, further comprising an antibacterial agent or an antifungal agent; wherein the antibacterial agent comprises tetracycline, doxycycline, or other antibiotics; wherein the antifungal agent comprises polyenes, Azoles—Imidazoles, Triazoles, Allylamines, Echinocandins, Griseofulvin, or tolnaftate.

12. The medical material of claim 1, wherein the medical material allows for local delivery of the animal or human protein, the anti-inflammatory agent, the antibacterial agent, or a combination thereof.

13. The medical material of claim 1, wherein the medical material forms a medical construct.

14. A medical construct fabricated from the medical material comprising an animal or human protein derived from a transgenic soybean or at least a portion of the transgenic soybean, wherein the transgenic soybean produces said animal or human protein.

15. The medical construct of claim 14, wherein the construct is for epidermal or dermal application.

16. The medical construct of claim 14, wherein the medical construct can be utilized for external or internal use in a subject; wherein the medical material is applied internally to an endoluminal tract of the subject, wherein the endoluminal tract comprises a gastrointestinal tract or a respiratory tract of the subject.

17. The medical construct of claim 14, wherein the material comprises a partially vacuous, discontinuous, or holey construct.

18. The medical construct of claim 14, wherein the medical construct is constructed by spin-coating, drop casting, spin casting, extrusion, electrospinning, film formation spraying, spray drying, drop casting, spin casting, extrusion, electrospinning, low-temperature thermoforming, micro-particle formation, nano-particle formation, micro-capsule formation, nano-capsule formation, or a combination thereof.

19. The medical construct of claim 14, wherein the medical construct is fabricated as a gauze, mesh, sheet, film, fibrous construct, or bandage.

20. A therapeutic medicament comprising a medical material comprising an animal or human protein derived from a transgenic soybean or at least a portion of the transgenic soybean, wherein the transgenic soybean produces said animal or human protein, wherein the medical material is a powder.


SEQUENCE LISTING (45 sequences)

SEQ ID NO: 1 - DNA, 180 bp, synthetic construct
  misc_feature 1..180: note = Modified EGF DNA sequence
  source 1..180: mol_type = other DNA
tctctttcttcagccgaaaattccgatagtgagtgtccactctcccatgatggctattgtttgcacgacggagtttgcatgtatattgaagctttggataagtacgcatgtaactgcgttgtgggatatatcggtgaaagatgccaatacagggacctcaaatggtgggagctgagataa

SEQ ID NO: 2 - DNA, 171 bp, synthetic construct
  misc_feature 1..171: note = Altered mod hEGF nucleaic acid sequence
  source 1..171: mol_type = other DNA
tcagccgaaacttccgatagtgagtgtccactctcccatgatggctattgtttgcacgacggagttcgcatgtatattgaagctttggataagtacgcatgtaactgcgttgtgggatatatcggtgaaagatgccaatacagggacctcaaatggtgggagctgagataa

SEQ ID NO: 3 - DNA, 171 bp, synthetic construct
  misc_feature 1..171: note = Altered modhEGF nucleic acid sequence
  source 1..171: mol_type = other DNA
tcagccgaaaactccgctagtgagtgtccactctcccatgatggctattgtttgcacgacggagttcgcatgtatattgaagctttggataagtacgcatataactgcgttgtgggatatatcggtgaaagatgccaatacagggacctcaaatggtgggagctgagataa

SEQ ID NO: 4 - DNA, 171 bp, synthetic construct
  misc_feature 1..171: note = Altered ModhEGF nucleic acid sequence
  source 1..171: mol_type = other DNA
tcagccgaaaactccgctagtgagtgttcactctcccatgatggcgattgtttgcacgacggagttcgcatgtatattgaagctttggataagtacgcatataactgcgttgtggaatatatcggtgaaagaggccaatacagggacctcaaacggtgggagctgagataa

SEQ ID NO: 5 - DNA, 171 bp, synthetic construct
  misc_feature 1..171: note = Altered modhEGF nucleic acid sequence
  source 1..171: mol_type = other DNA
tcagccgaaaactccgctattgagtgttcactctcccctgatggcgattgtttgcacgacggagttcgcatgtatattgaagctttgtataagtacgcatataactgcgttgtggaatatatcggtgaaagaggccaatacaggaacctcaaacggtgggagctgagataa

SEQ ID NO: 6 - DNA, 171 bp, synthetic construct
  misc_feature 1..171: note = Altered modhEGF nucleic acid sequence
  source 1..171: mol_type = other DNA
tcagccgaaaactccgctattgagtgttcactctcccctgatggcgattgtttgcaagacgtagttcgcatgtatagtgaagctttgtataagtacgcatataactgcgttgtggaatatctcggtgaaagaggccaatacaggaacctcaaacggtggaagctgagataa

SEQ ID NO: 7 - DNA, 171 bp, synthetic construct
  misc_feature 1..171: note = Altered modhEGF nucleic acid sequence
  source 1..171: mol_type = other DNA
tcagccgaaaatcccgatagtgagtgtccactctgccatgctggctattgttcgcacgacggagtttgcatgtatattgtagctgtggataagtacgcatgtaactgcgctgtgggatatatcggtgcaagatgccaatacagcgacctcaaatggtgggacccgagataa

SEQ ID NO: 8 - DNA, 171 bp, synthetic construct
  misc_feature 1..171: note = Altered modhEGF nucleic acid sequence
  source 1..171: mol_type = other DNA
tcagccgaaaatcccgatcgtgagtgtccactctgccatgctggctattgttcgcacgacggagtttgcatgtatattgtagctgtggataagtacgcatgtaactgcgctgtgggatatatcggtgcaagatgccaatacagcgacctcaaatggtgggacccgagataa

SEQ ID NO: 9 - DNA, 171 bp, synthetic construct; identical to SEQ ID NO: 8
  misc_feature 1..171: note = Altered modhEGF nucleic acid sequence
  source 1..171: mol_type = other DNA

SEQ ID NO: 10 - DNA, 171 bp, synthetic construct
  misc_feature 1..171: note = Altered modhEGF nucleic acid sequence
  source 1..171: mol_type = other DNA
tcagccgaaaatcccgatcgtgcgtgtccactctgccatgctgtctattgttcgcacgacggagtttgcatgtatattgtagctgtggataagtacgcatgtaactgcgctgtgggatatatcggtgcaagatgccaatacagcgacctcaaatggtgggacccgagataa

SEQ ID NO: 11 - DNA, 171 bp, synthetic construct
  misc_feature 1..171: note = Altered modhEGF nucleic acid sequence
  source 1..171: mol_type = other DNA
tcagccgaaaatcccgatcgtgcgtgtctactctgccatgctgtctattgttcgcacgacagagtttgcatgtatattgtagctgtggataagtactcatgtaactgcgctgtgggatgtatcggtgcaagatgccaatacagcgacctcaattggtgggagccgagataa

SEQ ID NO: 12 - protein, 59 aa, synthetic construct
  REGION 1..59: note = Modified EGF protein sequence
SLSSAENSDSECPLSHDGYCLHDGVCMYIEALDKYACNCVVGYIGERCQYRDLKWWELR

SEQ ID NO: 13 - protein, 59 aa, synthetic construct
  REGION 1..59: note = Altered modhEGF protein sequence
SLSSAETSDSECPLSHDGYCLHDGVRMYIEALDKYACNCVVGYIGERCQYRDLKWWELR

SEQ ID NO: 14 - protein, 59 aa, synthetic construct
  REGION 1..59: note = Altered modhEGF protein sequence
SLSSAETSASECPLSHDGYCLHDGVRMYIEALDKYAYNCVVGYIGERCQYRDLKWWELR

SEQ ID NO: 15 - protein, 59 aa, synthetic construct
  REGION 1..59: note = Altered modhEGF protein sequence
SLSSAETSASECSLSHDGDCLHDGVRMYIEALDKYAYNCVVEYIGERGQYRDLKRWELR

SEQ ID NO: 16 - protein, 59 aa, synthetic construct
  REGION 1..59: note = Altered modhEGF protein sequence
SLSSAETSAIECSLSPDGDCLHDGVRMYIEALYKYAYNCVVEYIGERGQYRNLKRWELR

SEQ ID NO: 17 - protein, 59 aa, synthetic construct
  REGION 1..59: note = Altered modhEGF protein sequence
SLSSAETSAIECSLSPDGDCLQDVVRMYSEALYKYAYNCVVEYLGERGQYRNLKRWKLR

SEQ ID NO: 18 - protein, 59 aa, synthetic construct
  REGION 1..59: note = Altered modhEGF protein sequence
SLSSAENADSECPLSHDGYCLHDGVCMYIVALDKYACNCVVGYIGERCQYRDLKWWELR

SEQ ID NO: 19 - protein, 59 aa, synthetic construct
  REGION 1..59: note = Altered modhEGF protein sequence
SLSSAENADRECPLSHAGYCLHDGVCMYIVALDKYACNCVVGYIGERCQYRDLKWWELR

SEQ ID NO: 20 - protein, 59 aa, synthetic construct
  REGION 1..59: note = Altered modhEGF protein sequence
SLSSAENADRECPLCHAGYCSHDGVCMYIVALDKYACNCAVGYIGERCQYSDLKWWEPR

SEQ ID NO: 21 - protein, 59 aa, synthetic construct
  REGION 1..59: note = Altered modhEGF protein sequence
SLSSAENADRACPLCHAVYCSHDGVCMYIVAVDKYACNCAVGYIGARCQYSDLKWWEPR

SEQ ID NO: 22 - protein, 59 aa, synthetic construct
  REGION 1..59: note = Altered modhEGF protein sequence
SLSSAENADRACLLCHAVYCSHDRVCMYIVAVDKYSCNCAVGCIGARCQYSDLNWWEPR

SEQ ID NO: 23 - DNA, 165 bp, Homo sapiens
  source 1..165: mol_type = other DNA
gtaagaaatagtgactctgaatgtcccctgtcccacgatgggtactgcctccatgatggtgtgtgcatgtatattgaagcattggacaagtatgcatgcaactgtgttgttggctacatcggggagcgatgtcagtaccgagacctgaagtggtgggaactgcgc

SEQ ID NO: 24 - protein, 39 aa, Homo sapiens
MKTNLFLFLIFSLLLSLSSAEFKTNLFLFLIFSLLLSLS

SEQ ID NO: 25 - protein, 5 aa, Homo sapiens
KHDEL

SEQ ID NO: 26 - DNA, 162 bp, synthetic construct
  misc_feature 1..162: note = Modified hEGF coding region
  source 1..162: mol_type = other DNA
aattccgatagtgagtgtccactctcccatgatggctattgtttgcacgacggagtttgcatgtatattgaagctttggataagtacgcatgtaactgcgttgtgggatatatcggtgaaagatgccaatacagggacctcaaatggtgggagctgagataa

SEQ ID NO: 27 - DNA, 162 bp, synthetic construct
  misc_feature 1..162: note = Altered modhEGF coding nucleic acid sequence
  source 1..162: mol_type = other DNA
acttccgatagtgagtgtccactctcccatgatggctattgtttgcacgacggagttcgcatgtatattgaagctttggataagtacgcatgtaactgcgttgtgggatatatcggtgaaagatgccaatacagggacctcaaatggtgggagctgagataa

SEQ ID NO: 28 - DNA, 162 bp, synthetic construct
  misc_feature 1..162: note = Altered modhEGF coding nucleic acid sequence
  source 1..162: mol_type = other DNA
aactccgctagtgagtgtccactctcccatgatggctattgtttgcacgacggagttcgcatgtatattgaagctttggataagtacgcatataactgcgttgtgggatatatcggtgaaagatgccaatacagggacctcaaatggtgggagctgagataa

SEQ ID NO: 29 - DNA, 162 bp, synthetic construct
  misc_feature 1..162: note = Altered modhEGF coding nucleic acid sequence
  source 1..162: mol_type = other DNA
aactccgctagtgagtgttcactctcccatgatggcgattgtttgcacgacggagttcgcatgtatattgaagctttggataagtacgcatataactgcgttgtggaatatatcggtgaaagaggccaatacagggacctcaaacggtgggagctgagataa

SEQ ID NO: 30 - DNA, 162 bp, synthetic construct
  misc_feature 1..162: note = Altered modhEGF cding nucleic acid sequence
  source 1..162: mol_type = other DNA
aactccgctattgagtgttcactctcccctgatggcgattgtttgcacgacggagttcgcatgtatattgaagctttgtataagtacgcatataactgcgttgtggaatatatcggtgaaagaggccaatacaggaacctcaaacggtgggagctgagataa

SEQ ID NO: 31 - DNA, 162 bp, synthetic construct
  misc_feature 1..162: note = Altered modhEGF coding nucleic acid sequence
  source 1..162: mol_type = other DNA
aactccgctattgagtgttcactctcccctgatggcgattgtttgcaagacgtagttcgcatgtatagtgaagctttgtataagtacgcatataactgcgttgtggaatatctcggtgaaagaggccaatacaggaacctcaaacggtggaagctgagataa

SEQ ID NO: 32 - DNA, 18 bp, Homo sapiens
  source 1..18: mol_type = other DNA
tctctttcttcagccgaa

SEQ ID NO: 33 - DNA, 18 bp, synthetic construct
  misc_feature 1..18: note = Altered nucleic acid sequence
  source 1..18: mol_type = other DNA
tctttttcttcagccgaa

SEQ ID NO: 34 - DNA, 18 bp, synthetic construct
  misc_feature 1..18: note = Altered nucleic acid sequence
  source 1..18: mol_type = other DNA
tctttttcttaagccgaa

SEQ ID NO: 35 - protein, 53 aa, Homo sapiens
NSDSECPLSHDGYCLHDGVCMYIEALDKYACNCVVGYIGERCQYRDLKWWELR

SEQ ID NO: 36 - protein, 11 aa, synthetic construct
  REGION 1..11: note = Protein extract from EGF transgenic soybean
CNCVVGYIGER

SEQ ID NO: 37 - DNA, 336 bp, synthetic construct
  misc_feature 1..336: note = Expression cassette
  source 1..336: mol_type = other DNA
gcggccgcccgatgaagactaatctttttctctttctcatcttttcacttctcctatcattatcctcggccgaattcaagactaacctgtttcttttcttgatttttagccttttgctctctctttcttcagccgaaaattccgatagtgagtgtccactctcccatgatggctattgtttgcacgacggagtttgcatgtatattgaagctttgaataagtacgcatgtaactgcgttgtgggatatatcggtgaaagatgccaatacagggacctcaaatggtgggagctgagatctgaaaagcatgatgaactttaatgagcggccgctaagt

SEQ ID NO: 38 - skipped

SEQ ID NO: 39 - DNA, 78 bp, synthetic construct
  misc_feature 1..78: note = ER directed 5 sequence
  source 1..78: mol_type = other DNA
ccgatgaagactaatctttttctctttctcatcttttcacttctcctatcattatcctcggccgaattcaagactaac

SEQ ID NO: 40 - DNA, 218 bp, synthetic construct
  misc_feature 1..218: note = Codon optimized for soybean the EGF protein
  source 1..218: mol_type = other DNA
ctgtttcttttcttgatttttagccttttgctctctctttcttcagccgaaaattccgatagtgagtgtccactctcccatgatggctattgtttgcacgacggagtttgcatgtatattgaagctttgaataagtacgcatgtaactgcgttgtgggatatatcggtgaaagatgccaatacagggacctcaaatggtgggagctgagatctgaaaa

SEQ ID NO: 41 - DNA, 19 bp, synthetic construct
  misc_feature 1..19: note = ER retention portion
  source 1..19: mol_type = other DNA
gcatgatgaactttaatga

SEQ ID NO: 42 - DNA, 20 bp, synthetic construct
  misc_feature 1..20: note = glycinin promoter primer
  source 1..20: mol_type = other DNA
tcattcaccttcctctcttc

SEQ ID NO: 43 - DNA, 20 bp, synthetic construct
  misc_feature 1..20: note = hygromycin gene primer HygF
  source 1..20: mol_type = other DNA
ctcactattcctttgccctc

SEQ ID NO: 44 - DNA, 20 bp, synthetic construct
  misc_feature 1..20: note = hygromycin gene primer HygR
  source 1..20: mol_type = other DNA
ctgacctattgcatctcccg

SEQ ID NO: 45 - protein, 4 aa, Glycine max
KDEL